(19) United States
(12) Patent Application Publication
(10) Pub. No.: US 2006/0026706 A1
(43) Pub. Date: Feb. 2, 2006

(54) Method for marker-less integration of a sequence of interest into the genome of a cell

(75) Inventors: Gilles Philippus Van Wezel, Oegstgeest (NL); Erik Vijgenboom, Berkel en Rodenrijs (NL)

Correspondence Address: TRASK BRITT, P.O. BOX 2550, SALT LAKE CITY, UT, 84110, US

(73) Assignee: Universiteit Leiden, Leiden (NL)

(21) Appl. No.: 11/140,625

(22) Filed: May 27, 2005

Related U.S. Application Data
(63) Continuation of application No. PCT/NL03/00835, filed on Nov. 27, 2003.

(30) Foreign Application Priority Data
Nov. 28, 2002 (EP) 02080000.9

(51) Int. Cl. A01H 1/00; C12N 15/82

(52) U.S. Cl. 800/278; 435/468

(57) ABSTRACT

Provided are multiple methods resulting in a site-specific, marker-less integration of a sequence of interest. The methods are based on the following: The genomic presence in a cell (host cell) of a selectable or screenable gene X. As a result of a mutation, this gene X can be essentially sensitive or insensitive to a certain component or condition Z or, as a result of a mutation in gene X, the host cell is made dependent on the presence of a certain component or condition Z; a plasmid on which the desired insertion (or deletion) is present, further harboring a truncated gene X and a selectable marker (such as an antibiotic-resistance gene) to select or screen for the presence or absence of vector sequences in the host cell; positive selection of the final recombination step, avoiding complicated and time-consuming screening for the desired recombinants. Preferably, both recombination steps are positively selected. When the starting host cell contains a mutant gene X on the genome, the final recombinant has a gene X without a mutation and the genome further only comprises the desired insertion or deletion. When the starting host cell contains an original gene X, the final recombinant bears a mutation in gene X and the genome further only comprises the desired insertion or deletion.

### CROSS-REFERENCE TO RELATED APPLICATIONS

This application is a continuation of PCT International Patent Application No. PCT/NL2003/000835, filed on Nov. 27, 2003, designating the United States of America, and published, in English, as PCT International Publication No. WO 2004/048580 A1 on Jun. 10, 2004, which application claims priority to European Patent Application Serial No. 02080000.9 filed on Nov. 28, 2002, the contents of each of which are hereby incorporated herein by this reference.

### TECHNICAL FIELD

The invention relates to the field of molecular biology, cell biology and biotechnology. More, in particular, the invention relates to an efficient method for obtaining site-specific, marker-less integration (or deletion) of a sequence of interest in (or from) the genome of a cell.

### BACKGROUND

Autonomously replicating plasmids are widely used for the introduction of desired DNA sequences into a host cell. However, stability is relatively low and plasmids are often lost rapidly when selection pressure is relieved. Furthermore, plasmid copy number varies per cell, resulting in low reproducibility. For these reasons, in biotechnology, the desired DNA is often inserted into the host genome by homologous recombination or by use of a host-specific integration system. This increases stability, while reproducibility is also higher.

Integration methods invariably require a marker on the delivery plasmid, such as an antibiotic-resistance gene or an antibiotic-free selectable marker, such as BADH (betaine aldehyde dehydrogenase, see, for example, PCT International Patent Publication WO 01/64023), which is unattractive from a biotechnological point of view, certainly if (part of) the production organism ends up in the food chain. Examples of the latter are genetically modified plants and vegetables or biomass of microbial origin (e.g., fungi) used in animal feeds. The use of chromosomal insertion by homologous recombination with concomitant loss of the vector sequences and its marker is, therefore, preferable.

After a certain recombination event, two possible recombinants can arise. The desired recombinant can either be selected or screened for. Selection is defined as having a way to force the presence of the desired situation, such as adding an antibiotic requires the presence of antibiotic resistance, while screening is defined as searching for a certain nonselectable characteristic, such as DNA sequencing, to establish the presence or absence of a change (insertion, deletion, mutation) in the genomic DNA.

One of the main problems with existing gene replacement protocols is that if there is no selectable marker left on the genome, this typically results in seemingly endless screening for the desired recombinants, without the certainty of success, making the process highly cost-ineffective and time consuming.

An overview of the various possibilities for gene inactivation strategies, with focus on actinomycetes, is given in Kieser et al. 2000. Gene replacement is a two-step procedure, with an insertion step and an excision event. While the insertion step can be selected due to the presence of a selectable marker on the plasmid, typically an antibiotic resistance marker, the second step often involves an indirect screening step (such as loss of the resistance cassette) unless a marker is left on the genome. However, the latter possibility is highly undesirable in biotechnological production strains.

Therefore, methods have been sought to achieve a more effective final recombination event. The main goal is, therefore, to find a way to avoid the need of screening in the last step, a process often involving replicating thousands of colonies to find a recombinant with the desired phenotype. One possible solution is to build in a counter-selectable marker into the delivery plasmid. For this purpose, the glkA gene has been used in streptomycetes by several laboratories (e.g., Buttner et al. 1990, van Wezel et al. 1995) and forms the basis for the disruption vector pIJ2581 (van Wezel and Bibb 1996). In such experiments, loss of the delivery plasmid is forced by growth on the glucose analogue 2-deoxyglucose, which is lethal for glkA+ strains. A disadvantage is that the host remains a glkA mutant, and this typically requires replacement by the wild-type glkA gene, for example, by crossing. Furthermore, unless a selectable marker, such as an antibiotic-resistance marker (or an antibiotic free selectable marker), is inserted into the target sequence on the genome, secondary recombinants still have to be screened for the correct recombination event, such as deletion or insertion of a DNA sequence. This is typically done by isolating genomic DNA from a large number of recombinants, followed by PCR and DNA sequencing, which is time consuming, and often the desired mutation or insertion is not found in the screen. A recent method designed to allow screening for deletions involves an initial gene replacement experiment whereby the DNA sequence to be deleted is replaced by a resistance marker, followed by recombinatory removal of the resistance marker, resulting in the desired deletion. This involves two cross-overs instead of one double, still without positive selection in the final step.

While such intensive screening programs to find the desired second recombination event are problematic in prokaryotes, growing hundreds of recombinant species to find a desired genotype without the aid of a selectable marker is often impossible in plants and other higher eukaryotes. For this reason, in plant biotechnology, scientists resort to the unfavorable solution of leaving a selectable marker (such as bialaphos or kanamycin-resistance) on the genome of the recombinant.

In recent years, the use of selectable marker genes for the identification of recombinant microorganisms has become subject to intensive debate. The spread of such marker genes into the environment is highly undesirable and even more so if these marker genes encode resistance against antimicrobial agents. Therefore, other markers (such as BADH) have been developed to reduce this environmental risk, although their use is also subject to restraints. Therefore, ideally, one would like to produce recombinant organisms that do not contain any additional DNA other than the DNA of interest. An example of a method for marker-free integration of DNA into the genome of microorganisms (fungi and bacteria) is presented in European patent application 0 635 574 A1. In this case, the DNA of interest is cloned on a vector between two identical DNA sequences and, after integration of the plasmid into the host genome, the inventors select for a second recombination event, removing all vector sequences other than the DNA of interest and a single element of the repeat sequence. However, while such a method results in a marker-free recombinant strain, still a significant section of additional (and in principle undesired) DNA is left in the host genome, typically with a minimum length of several hundreds of base pairs. Furthermore, vector systems with repeat sequences typically have reduced stability due to possible recombination events between the repeats during plasmid replication.

There is, therefore, a clear need for an integration (or deletion) method in which all steps are selectable and particularly, the final recombination event, where the only change to the host genome is (preferably) the desired insertion (or deletion), without leaving a selection marker (e.g., an antibiotic resistance gene) or a mutant host (e.g., a glucokinase mutant in bacteria or an ethylene receptor mutant in plants).

### SUMMARY OF THE INVENTION

Disclosed is a reliable and highly effective method for inserting (or deleting) a sequence of interest (preferably DNA) into (or from) the host genome, which is, for example, used for the insertion of an expression cassette for enzyme production. The method is easily translated to any organism, provided that the suitable criteria for the organism are met. For example, the choice of selection marker (which is used in the method according to the invention but which is later removed) is adapted to the organism. Upon reading the detailed description of the present invention, it will be clear to a person skilled in the art that the invention is applicable to a broad range of hosts (cells), as the only principal requirement is the availability of a transformation procedure and a selectable (or screenable) host gene.

Provided are multiple methods resulting in a site-specific, markerless integration (or deletion) of a sequence of interest. The methods are based on the following principles:

The genomic presence in a cell (host cell) of a selectable or screenable gene X. As a result of a mutation, this gene X can be essentially sensitive or insensitive to a certain component or condition Z or, as a result of a mutation in gene X, the host cell is made dependent on the presence of a certain component or condition Z.

A plasmid on which the desired insertion (or deletion) is present, further harboring a truncated version of gene X and a selectable marker (such as an antibiotic-resistance or an antibiotic-free selectable marker gene) to select or screen for the presence or absence of vector sequences in the host cell.

Positive selection of the final recombination step, avoiding complicated and time-consuming screening for the desired recombinants. In a preferred situation, both recombination steps can be positively selected.

When the starting host cell contains a mutant gene X on the genome, the final recombinant has a gene X without a mutation and, preferably, the genome further only comprises the desired insertion or deletion. When the starting host cell contains an original gene X, the final recombinant bears a mutation in gene X and (preferably) the genome further only comprises the desired insertion or deletion.

Some of the possibilities are exemplified in FIGS. 1 through 4. After recombination of these genomes with a plasmid that comprises a truncated version of gene X and a sequence of interest and after performing several selection steps (for example, based on the presence of a selection marker), a cell is obtained in which the sequence of interest is integrated (or deleted) via the process of homologous recombination, without the final presence of a selection marker.

Table 1 discloses an overview of selection criteria that can be applied in the recombination scheme as depicted in FIGS. 1 through 4. Table 2 discloses a non-limiting list of examples of the “gene X.” It is clear that based on the information provided in the figures and tables, different combinations are easily made by a person skilled in the art without deviating from the spirit of the present invention, which all rely on the fact that the final recombination step in a method for site-specific, marker-less integration or deletion of a sequence of interest is selectable.

Preferably, gene X is an endogenous gene. As a result of a mutation, gene X can be essentially sensitive or insensitive to a certain component or condition Z or, as a result of a mutation in gene X, the host cell is made dependent on the presence of a certain component or condition Z. The term “gene X” is not restricted to the sequence encoding the open reading frame of the corresponding protein, but typically also comprises the necessary sequences for proper transcription and translation, in particular, promoter and/or termination sequences as well as the ribosome-binding site.

The invention provides in one embodiment, a method for obtaining site-specific, marker-less integration of a sequence of interest in the genome of a cell, wherein the genome comprises a selectable or screenable gene X and a sequence Y, the method comprising:
providing the cell with a plasmid which comprises:
   a truncated version of gene X;
   a substantial part of sequence Y;
   a sequence of interest located between the truncated version of gene X and sequence Y;
   a selection marker located outside the sandwich of the truncated version of gene X, the sequence of interest and sequence Y;
selecting for a first recombination event by using the selection marker of the plasmid, thereby obtaining a cell in which the plasmid has integrated via homologous recombination into the genome of the cell;
selecting or screening for the selectable or screenable gene X, thereby obtaining a cell with a recombinant genome in which recombination has occurred through sequence Y of the genome and sequence Y of the plasmid; and
selecting positively for a second recombination event, thereby obtaining a cell with a recombinant genome in which an internal recombination event has occurred through gene X and the truncated version of gene X.

The positive selection for the second recombination event for obtaining a cell with a recombinant genome in which an internal recombination event has occurred through gene X and the truncated version of gene X is based on the presence of the final desired genomic version of gene X. This is explained in more detailed hereunder.

In a preferred embodiment, the invention provides a method wherein the plasmid essentially cannot replicate during the first recombination event. Hence, when a selection step is performed for the presence of the selection marker of the plasmid, only cells in which the genetic information from the plasmid has been integrated in the genome will survive. Preferably, the plasmid can replicate in the cell to multiple copies after the transfer of the plasmid to the cell and replication is blocked during the first recombination event. Such a feature is, for example, obtained by providing the plasmid with a conditionally dependent ori. This increases the efficiency of the first recombination event.

In yet another preferred embodiment the invention provides a method that further comprises a check after the second recombination event for loss of the selection marker of the plasmid. This is easily performed by comparing the growth of, for example, bacterial colonies on plates with and without the corresponding antibiotic. In yet another preferred embodiment, the invention provides a method wherein the obtained cell in which an internal recombination event has occurred through gene X and the truncated version of gene X is checked for the presence of the sequence of interest, for example, PCR analysis followed by sequence analysis.

In a preferred embodiment, the invention comprises a method, wherein the selectable or screenable gene X is selectable or screenable via a component or a chemical and/or physical condition or wherein the cell is dependent on the presence of the component or condition due to the presence of the selectable or screenable gene X. Examples of suitable combinations of gene X and component or condition will be outlined below. Some examples of a component or a chemical and/or physical condition are temperature, light, H₂O₂, vitamins and amino acids.

In another preferred embodiment, the invention provides a method wherein the truncated version of gene X is inactive through truncation but otherwise original (for example, as illustrated in FIGS. 1 and 2). It is clear from FIG. 3 that the truncated version of gene X can also be an inactive (due to the truncation) and mutated (hence, otherwise non-functional) version of gene X. Preferably, the mutation comprises a point mutation.

In an even more preferred embodiment, the invention provides a method, wherein the final recombinant has, except for the desired insertion, an original genome (more specifically, with an original gene X) and, even more preferably, a method wherein both recombination steps are selectable. However, it is clear from FIG. 3 that it is also possible to obtain a final recombinant that comprises a mutation (for example, a point mutation) in gene X. Use of the latter is, for example, acceptable in a laboratory strain or under production under non-GMP conditions.

The term “original” is herein used to refer to the starting situation before a method according to the invention, possibly preceded by a method for preparing the cell in which the site-specific, marker-less integration or deletion must take place, is applied. For example, when gene X is glkA (encoding glucose kinase), the original genome comprises a glk gene, which results in a sensitive phenotype of the cell to 2-deoxyglucose (2-DOG). First, a mutant of the glk gene is produced (by methods known to a person skilled in the art), which mutant renders the cell insensitive to 2-DOG (hence, a selectable gene X is obtained). Then, a method according to the invention is applied and after the final recombination event, the resulting cell will comprise a glk gene that renders the cell (again) sensitive for 2-DOG. It is clear that this logic can be applied mutatis mutandis to the other examples of gene X herein disclosed.

The method according to the invention can be carried out with different types of gene X and non-limiting examples are disclosed herein.

In one of the embodiments, gene X is mutated such that it is essentially insensitive to a certain component or condition Z and, hence, the invention provides a method for obtaining site-specific, marker-less integration of a sequence of interest in the genome of a cell, wherein the genome comprises a gene X, which, as a result of a mutation, is essentially insensitive to a certain component or condition Z, the genome further comprising a sequence Y, the method comprising:
providing the cell with a plasmid which comprises:
   a truncated inactive but otherwise original version of gene X;
   a substantial part of sequence Y;
   a sequence of interest located between the truncated inactive but otherwise original version of gene X and sequence Y;
   a selection marker located outside the sandwich of the truncated inactive but otherwise original version of gene X, the sequence of interest and sequence Y;
selecting for a first recombination event by using the selection marker of the plasmid, thereby obtaining a cell in which the plasmid has integrated via homologous recombination into the genome of the cell;
selecting or screening for gene X, which is essentially insensitive to a certain component or condition Z, by using component or condition Z, thereby obtaining a cell with a recombinant genome in which recombination has occurred through sequence Y of the genome and sequence Y of the plasmid; and
selecting positively for a second recombination event via component or condition V (defined hereunder), thereby obtaining a cell with a recombinant genome in which the second recombination event has occurred internally through the sequences of gene X and the truncated inactive but otherwise original version of gene X.

The choice for condition V is based on the final (desired) outcome of the genomic version of gene X. Hence, this choice is based on the characteristics/properties of the genomic version of gene X (in the host cell) before the first recombination event through sequence Y of the genome and sequence Y of the plasmid and after a second recombination event through the sequences of gene X and the truncated inactive but otherwise original version of gene X. The choice for condition V will be exemplified in more detail at the different discussions on the figures.

In a preferred embodiment, sequence Y of the genome is located downstream of gene X, which, as a result of a mutation, is essentially insensitive to a certain component or condition Z and wherein the plasmid comprises:
a 5′ truncated inactive but otherwise original version of gene X;
a sequence of interest located downstream of the 5′ truncated inactive but otherwise original version of gene X;
a substantial part of sequence Y located downstream of the sequence of interest;
a selection marker located outside the sandwich of the 5′ truncated inactive but otherwise original version of gene X, the sequence of interest and sequence Y; and
wherein the second recombination event has occurred internally through the sequences upstream of the mutation in gene X and the 5′ truncated inactive but otherwise original version of gene X. This is exemplified by FIG. 1A.

FIG. 1A discloses a schematic overview of a method according to the invention, where the particular combination of DNA sequences was designed for use in actinomycetes and preferably in streptomycetes. This overview is exemplified by the use of (i) a glucose kinase (glkA) mutant as a gene X, which, as a result of a mutation, is essentially insensitive to a certain component or condition Z, (ii) the sequence directly downstream of the sequence of interest is referred to as sequence Y (in this particular example, sequence Y encompasses an open reading frame, but this is not necessary, sequence Y may also consist of “non-coding” sequences or a combination of coding and non-coding sequences); sequence Y on the genome corresponds to (preferably, is identical to) sequence Y on the plasmid, (iii) cloned DNA as sequence of interest (preferably comprising an open reading frame encoding a protein of interest and sequences required for proper transcription and/or translation, i.e., promoter and/or terminator sequences, (iv) the thiostrepton-resistance gene (tsr) as a selection marker present on the plasmid, (v) 2-deoxyglucose as component or condition Z, (vi) glucose as component or condition V and a 5′ truncated gene X on the plasmid. In a first step, the plasmid is transferred to the cell of interest, for example, by electroporation, protoplast transformation, transfection, transduction or any other known method. In a preferred embodiment, the plasmid essentially cannot replicate during the first recombination event and, hence, when a selection step is performed for the presence of tsr, only cells in which the genetic information from the plasmid has been integrated in the genome will survive. It is clear from FIG. 1A that there are two major possible recombination events. In the first one (designated 1 in FIG. 1A), recombination has occurred between the (sequence of the) glkA mutant on the genome and the (sequence of the) 5′ truncated inactive but otherwise original version of glkA on the plasmid. This results in the presence of a complete and expressed wild-type glkA gene and, hence, this recombinant is sensitive to 2-deoxyglucose (further designated as 2-DOG). In the second possible recombination (designated 2 in FIG. 1A), recombination has occurred between the 3′ ORF sequences present in the genome and in the plasmid. This results in the presence of a complete and expressed mutant glkA gene that is essentially insensitive to 2-DOG. Hence, selection of the recombinants on 2-DOG results in a selection for recombinant 2. A second (internal) recombination event then selects for recombination via the sequences upstream of the mutation in glkA and the 5′ truncated inactive but otherwise original version of glkA of the plasmid. The screening of this further, internal recombination event is performed via positive selection via component or condition V, in this example, glucose. The final recombinant has a wild-type glkA gene and, hence, is capable of growing on glucose. On the other hand, strains comprising the mutated glkA gene cannot grow on the glucose as sole carbon source. Optionally, the final recombinant is then checked for its sensitivity to 2-DOG and tsr and the presence of the sequence of interest is optionally confirmed by, for example, PCR analysis and/or by sequence analysis.

The opposite situation, with selectable gene X downstream instead of upstream of sequence Y, is also possible (FIG. 1B). Again, the desired insertion is positioned between sequences X and Y. In this case, gene X (on the plasmid) is truncated at the 3′ end, and the mutation rendering the gene product insensitive to the selection criterion lies preferably at the front (5′ end) of the gene. In this case, sequence Y corresponds to the sequence directly upstream of the gene of interest/cloned gene.

Any sequence can be used as the sequence of interest. Preferably, the sequence enables the production of a product/protein of interest not present as such or present in low concentrations in the cell. Hence, the sequence of interest preferably also comprises the necessary elements for proper transcription and/or translation (such as functionally linked promoter and terminator sequences), for example, a sequence specifying an enzyme, an enzyme inhibitor, an antitumor agent, a bioinsecticide, a part of an antibody (for example, a heavy chain or a light chain), or an anti-migraine agent. However, it should be kept in mind that not only sequences can be inserted according to a method of the invention, but also deletions can be introduced. Examples of the creation of deletions upstream or downstream of a selectable or screenable gene X are shown in FIGS. 4A and 4B, respectively. In this case, the second step is selectable while the first desired recombination event is not always selectable. Again, this methodology avoids the currently routine method of randomly picking many recombinant organisms and checking their genomic DNA to identify possible correct recombinants, if they are present at all. Hence, the invention also provides a method for obtaining site-specific, marker-less deletion of a sequence of interest from the genome in a cell, wherein the genome comprises a selectable or screenable gene X and a sequence Y, the method comprising the herein disclosed steps. In this particular case, the sequence of interest is such that the method results in a deletion.

As used herein, the term “vector” is used to indicate a so-called empty (without any extra sequences) cloning vector (for example, pUC18 or pBR322). The term “plasmid” is used to refer to a vector in which a sequence has been cloned (for example, a sequence of interest). Hence, in the final step of a method according to the invention, it is checked whether all vector-related sequences have been removed from the genome, leaving only the sequence of interest behind. The removal of the vector sequences is, for example, determined by screening for loss of the selection marker.

The strategy for constructing the plasmid is briefly described. First, the desired site-specific genomic location for insertion (or deletion) upstream or downstream of a selectable or screenable gene X is determined. This position is preferably chosen such that the promoter and/or terminator can provide (proper) transcription, while translational signals such as the ribosome-binding site are also left intact. In the situation described in FIG. 1A, this site is preferably chosen approximately 50 bp (in case of bacterial situations) downstream of the stop codon of gene X. In the case of FIG. 1B, this site would be typically chosen approximately 100-200 bp upstream of the start codon of gene X, or as much as is required to leave the regulatory elements for gene X intact (again in bacterial situations). The skilled person can easily determine by standard methods which sequences are necessary for a proper/acceptable transcription and/or translation and, hence, the person skilled in the art can also easily determine the proper distances from gene X for insertion (or deletion). With regard to sequence Y, the following is noted: in principle, sequence Y can be any sequence (directly) upstream (FIG. 1B) or downstream (FIG. 1A) of the site chosen for site-specific integration (or deletion). Preferably, it is chosen (by the skilled person) in such a way that the distance between gene X and sequence Y allows efficient integration (or deletion) by the process of (preferably homologous) recombination.

Any common selection marker can be used to identify the presence of vector sequences. The person skilled in the art will know how to select the proper selection marker for each cell type. For example, ampicillin, apramycin or kanamycin for an E. Coli cell, apramycin, hygromycin or kanamycin for a streptomycete, or kanamycin, cyanamid or hygromycin for a plant cell.

The plasmid used in the method according to the invention further comprises all necessary elements for cloning and propagation in a host other than the host that is the target for the chromosomal insertion or deletion, for example; an origin of replication (ori) enabling the production or maintaining of the plasmid in E. coli. The person skilled in the art is very well capable of selecting all the necessary elements and a detailed discussion on this item is, therefore, not provided.

In a preferred embodiment, gene X, which, as a result of a mutation, is essentially insensitive to a certain component or condition Z, is mutated close to the 3′ end of gene X in the situation outlined in FIG. 1A, or mutated close to the 5′ end of gene X in the situation outlined in FIG. 1B. The presence of the mutation close to the end of the gene ensures maximal efficiency for the second recombination, which results in a desired gene X after the final recombination step. It is clear to a person skilled in the art that such a mutation can be a point mutation, a small deletion, or even a small insertion.

When the starting host cell contains a mutant gene X on the genome, the final recombinant has a gene X without a mutation and the genome further only comprises the desired insertion or deletion. When the starting host cell contains an original gene X, the final recombinant bears a mutation in gene X and the genome further only comprises the desired insertion or deletion.

In a preferred embodiment, the method according to the invention comprises a screening step after the first recombination event to rule out that recombination has occurred upstream of the cloned DNA, but downstream of the site of mutation (see FIG. 5, situation 1B). The probability of such a recombination event can be calculated with the formula provided in the experimental part herein and, hence, the necessity of such an extra step can also be based on this formula. In the glkA experiment described in the experimental part, where the ratio A:B is around 5:2, all three colonies checked had undergone recombination through area A.

The length of gene X is not critical. In principle, such a gene will be at least several 100 bp because it encodes a protein that is essentially insensitive to a certain component or condition Z. However, it is clear to a person skilled in the art that, in general, the frequency of the recombination increases with increasing lengths of gene X and sequence Y. With regard to applications in actinomycetes and especially in streptomycetes, the lengths of gene X and sequence Y are preferably at least 400 to 500 bp to ensure an acceptable frequency. A person skilled in the art is very well capable of selecting, based on the cell in which the integration must take place, a suitable length of gene X. For example, for use of a method according to the invention in E. coli, the length of gene X can be reduced well below 100 bp.

In principle, it is possible to use non-homologous DNA sequences for recombination, although recombination frequencies are strongly reduced. Several examples are given in Kieser et al. (2000). However, to get recombination between heterologous genes at a reasonable frequency, more than 95% identity between the genes or regions in which recombination is desired, is highly desirable, and certainly when there is no positive selection, such as in the final step of most double cross-over events. Hence, in a preferred embodiment, the sequence Y on the genome and sequence Y on the plasmid are at least 95% identical. The same is true for gene X on the genome and gene X on the plasmid.

In an even more preferred embodiment, the mutation in gene X comprises a point mutation and, more preferably, a point mutation at the 3′ or 5′ end which ensures a final recombinant with an original gene X.

In yet an even more preferred embodiment, the invention provides a method according to the invention wherein the substantial part of sequence Y located downstream of the sequence of interest is approximately of the same length as the 5′ truncated inactive but otherwise original version of gene X, to improve the probability of the desired second recombination event. A substantial part is herein defined as a part that is capable of providing recombination. The length and overall homology depends on the cell used. For example, recombination in a hyperrecombinant E. coli strain can take place with sequences as small as 40 bp. Recombination in streptomycetes typically involves sequences of at least 400 bp. A person skilled in the art knows how to select the proper length and, hence, no further details are provided.

It is clear from the description in FIG. 1A that one possible combination of a gene X which is essentially insensitive to a certain component or condition Z and a component or condition Z is mutated glkA and 2-deoxy-glucose. Even more preferred, mutated glkA is mutated as depicted in FIG. 8. Other examples of suitable glkA mutants are disclosed in Table 2. In principle, every mutant of glkA that results in the ability to grow on 2-DOG can be used in a method according to the invention. Mutants in the 5′ end of glkA in a method as exemplified in FIG. 1B and mutants in the 3′ end of glkA in a method as exemplified in FIG. 1A. Mutants that comprise a mutation somewhere in the middle of the glkA gene may also be used, but their use will result in lower frequencies of final desired recombinants (see also, explanation on FIG. 5). The use of a mutated glkA as a gene X in the genome and 2-DOG as component or condition Z can be applied to all bacterial cell types, because all prokaryotes comprise a functional homologue of the glkA gene, which is responsible for the conversion of glucose to glucose-6-phosphate.

In principle, every gene whose wild-type product confers sensitivity to a certain component or condition Z can be applied in a method according to the invention. All that is preferably needed is a genomic mutant of the gene, preferably with the mutation close to the 3′ end of the gene (for the situation as depicted in FIG. 1A), wherein the mutant is essentially insensitive to a certain component or condition Z. For example, a glkA mutant is obtained by growing wild-type strains on 2-DOG-containing media and selecting for ability to grow on this medium. The glkA mutants can be further identified by, for example, sequence analysis and, hence, a mutant mutated at the 3′ end is obtained.

In a preferred embodiment, gene X is followed by a transcriptional terminator on the genome and insertion of the sequence of interest has no effect on the proper transcription and/or translation of downstream-located genes. This avoids polar effects on downstream-located genes. A detailed analysis of these problems and ways to avoid them are outlined in the experimental part.

Another example of such a combination of a gene X that is essentially insensitive to a certain component or condition Z and sensitive to a component or condition V, is mutated rpsL (encoding r-protein S12) and streptomycin. In this special case, both V and Z are streptomycin. Several streptomycin-dependent mutants are known in prokaryotes (Timms et al. 1992), which require streptomycin for growth due to strongly enhanced accuracy of translation in these mutants, which is counteracted by streptomycin. Replacing glkA by rpsL in FIG. 1A or 1B, the first desired recombination event is selected in the presence of streptomycin, while the final recombination event is selected by removing streptomycin. Similar to glucokinase, ribosomal protein S12 occurs in all known prokaryotes and application is, therefore, possible in a very broad range of hosts.

Yet another example of such a combination of a gene X that is essentially insensitive to a certain component or condition Z and sensitive to a component or condition V, which is applicable in plants, is a mutated gene for the ethylene receptor protein 1 (ERP1). An alignment of EPR1 homologues from various plants is shown in FIG. 9. Mutant seedlings grow much faster than original seedlings in the presence of ethylene, providing positive selection for the mutation, while positive selection of original plants is possible on the basis of much faster germination, enhanced peroxidase production, and reduced chlorophyll production (Bleecker et al. 1988). In the first recombination step, situation 2 is selected on the basis of (enhanced) growth in the presence of ethylene. Original plants generated in the desired final recombination event are characterized on the basis of fast germination, less green leaves, and the anticipated higher peroxide resistance.

In yet another embodiment, gene X is mutated such that the (host) cell requires the presence of a component or condition Z and, hence, the invention provides a method for obtaining site-specific, marker-less integration of a sequence of interest in the genome of a cell, wherein the genome comprises a mutated gene X and wherein the cell is, due to the mutated gene X, dependent on the presence of a certain component or condition Z, the genome further comprising a sequence Y, the method comprising:
providing the cell with a plasmid which comprises:
   a truncated inactive but otherwise original version of gene X;
   a substantial part of sequence Y;
   a sequence of interest located between the truncated inactive but otherwise original version of gene X and sequence Y;
   a selection marker located outside the sandwich of the truncated inactive but otherwise original version of gene X, the sequence of interest and sequence Y;
selecting for a first recombination event by using the selection marker of the plasmid, thereby obtaining a cell in which the plasmid has integrated via homologous recombination into the genome of the cell;
selecting or screening for a recombinant cell which requires the presence of component or condition Z, thereby obtaining a cell with a recombinant genome in which recombination has occurred through sequence Y of the genome and sequence Y of the plasmid; and
selecting positively for a second recombination event by identifying a recombinant cell that does not require the presence of component or condition Z, thereby obtaining a cell with a recombinant genome in which the second recombination event has occurred internally through the sequences of gene X and the truncated inactive but otherwise original version of gene X.

In a preferred embodiment, sequence Y of the genome is located downstream of the mutated gene X, wherein the plasmid comprises:
a 5′ truncated inactive but otherwise original version of gene X;
a sequence of interest located downstream of the 5′ truncated inactive but otherwise original version of gene X;
a substantial part of sequence Y located downstream of the sequence of interest;
a selection marker located outside the sandwich of the 5′ truncated inactive but otherwise original version of gene X, the sequence of interest and sequence Y; and
wherein the second recombination event has occurred internally through the sequences upstream of the mutation in gene X and the 5′ truncated inactive but otherwise original version of gene X.

Cells or host cells for use in such a method are readily obtainable by, for example, classical mutagenesis methods known by the person skilled in the art.

This part of the invention is illustrated in FIG. 2. It is clear to a person skilled in the art that, similarly to the situations illustrated in FIGS. 1A, 1B and 2, the combination of a 3′ truncated gene X with a 5′ located sequence Y is possible, again with the mutation in gene X situated preferentially close to the start of the gene. Therefore, this method is not explained in more detail.

Preferably, the mutated gene X is a mutated amino acid biosynthesis gene and component or condition Z is the corresponding amino acid. In another preferred embodiment, the mutated gene X is a mutated vitamin biosynthesis gene and component or condition Z is the corresponding vitamin.

Such cells or host cells are readily obtainable by, for example, classical mutagenesis methods known by the person skilled in the art.

This part of the invention is exemplified by the use of auxotrophic markers. Auxotrophy is the inability of, in general, microorganisms to synthesize certain compounds, such as amino acids, from precursors. In contrast to corresponding wild-type strains, auxotrophic variants do not grow on so-called minimal media. Auxotrophic strains only grow on minimal media supplemented with the required growth factors, such as vitamins and/or amino acids.

This part of the invention is exemplified in FIG. 2 and, in a first step, a plasmid comprising a sequence of interest and a 5′ truncated inactive but otherwise original version of an amino acid biosynthesis gene is transferred to a cell of interest. The cell of interest comprises a genomically located mutated amino acid biosynthesis gene and, hence, essentially requires the presence of the corresponding amino acid, in the absence of which, the cell fails to grow. In a preferred embodiment, the plasmid cannot replicate during the first recombination event and only those that have integrated the plasmid into the genome will survive. Again, two major recombination events are possible. In the first possibility, recombination has occurred between the mutant amino acid biosynthesis gene on the genome and the 5′ truncated inactive but otherwise original version of the amino acid biosynthesis gene on the plasmid. This results in the presence of a complete and expressed functional amino acid biosynthesis sequence and, hence, this recombinant does not require the corresponding amino acid for growth. In the second possibility, recombination has occurred between the sequences Y present on the genome and on the plasmid. This results in the presence of a complete and expressed mutant amino acid biosynthesis gene sequence and in a recombinant organism that requires the corresponding amino acid for growth.

Recombinants obtained via the second possible recombination are screened, for example, by comparing the growth of recombinants in the presence or absence of the corresponding amino acid. In a further recombination event, selection is made for recombination between the sequence upstream of the mutation in the chromosomally located (mutant) copy of the amino acid biosynthesis gene and the 5′ truncated inactive but otherwise original version of the amino acid biosynthesis gene. These final recombinants are then selected by their ability to grow on media which do not contain the corresponding amino acid and, optionally, screened for absence of the selection marker of the plasmid. Also optionally, the presence of the sequence of interest is confirmed by, for example, a PCR and/or sequence analysis.

This method provides a positive selection step for identifying the desired final recombinants and, hence, the success rate of identifying a final desired recombinant is optimized, avoiding failed experiments, and experimental time and effort reduced significantly.

In principle, every gene whose original product confers the ability to grow without the need for amino acids, vitamins and other essential building blocks can be applied in a method as described above. All that is required is an endogenous gene located on the genome, with a mutation at either end of the gene, making the cell dependent on a certain component or condition Z.

In yet another embodiment, the genome comprises a gene X, which, as a result of a mutation, is essentially sensitive to a component or condition Z and, hence, the invention provides a method for obtaining site-specific, marker-less integration of a sequence of interest in the genome of a cell, wherein the genome comprises a gene X, which, as a result of a mutation, is essentially sensitive to a certain component or condition Z, the genome further comprising a sequence Y, the method comprising:
providing the cell with a plasmid which comprises:
   a truncated inactive but otherwise original version of gene X;
   a substantial part of sequence Y;
   a sequence of interest located between the truncated inactive but otherwise original version of gene X and sequence Y;
   a selection marker located outside the sandwich of the truncated inactive but otherwise original version of gene X, the sequence of interest and sequence Y;
selecting for a first recombination event by using the selection marker of the plasmid, thereby obtaining a cell in which the plasmid has integrated via homologous recombination into the genome of the cell;
screening for a recombinant cell which is sensitive to a certain component or condition Z, thereby obtaining a cell with a recombinant genome in which recombination has occurred through sequence Y of the genome and sequence Y of the plasmid; and
selecting positively for a second recombination event by identifying a recombinant cell which is insensitive to component or condition Z, thereby obtaining a cell with a recombinant genome in which an internal recombination event has occurred through the sequences of gene X and the truncated inactive but otherwise original version of gene X.

In a preferred embodiment, the invention provides a method, wherein sequence Y of the genome is located downstream of gene X, which, as a result of a mutation, is essentially sensitive to a certain component or condition Z and wherein the plasmid comprises:
a 5′ truncated inactive but otherwise original version of gene X;
a sequence of interest located downstream of the 5′ truncated inactive but otherwise original version of gene X;
a substantial part of sequence Y located downstream of the sequence of interest;
a selection marker located outside the sandwich of the 5′ truncated inactive but otherwise original version of gene X, the sequence of interest and sequence Y; and
wherein the second recombination event has occurred internally through the sequences upstream of the mutation gene X and the 5′ truncated inactive but otherwise original version of gene X.

Again, it is clear to a person in the art that, similarly to the situations illustrated in FIGS. 1A, 1B and 2, the combination of a 3′ truncated gene X with a 5′ located sequence Y is possible, again with the mutation in gene X situated preferentially close to the start of the gene. Therefore, this method is not explained in more detail.

Preferably, gene X, which, as a result of a mutation, is essentially sensitive to a certain component or condition Z, is a mutated peroxidase or catalase gene and component or condition Z is H₂O₂. Another example of a gene X, which, as a result of a mutation, is essentially sensitive to a certain component or condition Z, is a gene which is sensitive to a certain antibiotic and that becomes resistant after the final recombination event. Hence, in another preferred embodiment, gene X, which, as a result of a mutation, is essentially sensitive to a certain component or condition Z, is a mutated β-lactamase and component or condition Z is a β-lactam-antibiotic. Yet another example of a gene X, which, as a result of a mutation, is essentially sensitive to a certain component or condition Z, is a gene that is sensitive to elevated or reduced temperatures, known as heat-shock or cold-shock conditions, respectively.

Cells or host cells for use in such a method are readily obtainable by, for example, classical mutagenesis methods known by the person skilled in the art.

This method is exemplified by the use of catalase as a gene X that is as a result of a mutation essentially sensitive to H₂O₂ and proceeds through the following steps (see FIG. 2). In the first step, the plasmid comprising a 5′ truncated inactive but otherwise original version of the catalase gene and a sequence of interest, is transferred to the cell of interest. The genome of the cell comprises a catalase gene, which, as a result of a mutation, is essentially inactive, rendering the cell sensitive to H₂O₂. Preferably, the plasmid essentially cannot replicate during the first recombination event and only those that have integrated the plasmid into the genome will survive. Again, two major recombination events are possible. In the first event, recombination has occurred between the mutant catalase gene on the genome and the 5′ truncated inactive but otherwise original version of the catalase gene on the plasmid. This results in the presence of a complete and expressed functional catalase gene and, hence, this recombinant is insensitive to H₂O₂. In the second recombination event, recombination has occurred between the sequences Y present on the genome and on the plasmid. This results in the presence of a complete and expressed mutant catalase gene that is essentially inactive, rendering the cell sensitive to H₂O₂. The second possibility is screened for. In a further recombination event, a selection is made for a recombination event via the sequences upstream of the mutation in the mutant chromosomally located copy of the catalase gene and the 5′ truncated inactive but otherwise original version of the catalase gene. This recombinant in which a second recombination event has occurred is then selected by its insensitivity to H₂O₂ and, hence, the final step is performed on the basis of positive selection criteria. Optionally, the presence of the sequence of interest is confirmed by, for example, PCR followed by sequence analysis.

Examples of genes which can be used in this part of the invention are katG (E. coli, Synechocystis PCC6803), cpeB (S. coelicolor).

Another example of a gene X, which, as a result of a mutation, is essentially sensitive to a certain component or condition Z, is a thermosensitive (Ts) mutation that renders the (host) cell sensitive to higher temperatures. While this does not allow positive selection for one of the two alternative recombination events (1 and 2 in FIG. 2), positive selection remains in the crucial final recombination step, for example, by reversing a Ts mutation to allow growth at higher temperatures. The possible use of Ts mutants is very attractive, since (1) Ts mutations can be introduced in many, if not all, essential genes, making the system universally applicable, and (2) the final step is by far the most difficult and time consuming in terms of screening.

In the case a gene X is used that is sensitive to component or condition Z, another way of providing the corresponding truncation is by providing a transciptionally silent gene X, for example, by creating a mutation in a crucial part of the promoter consensus sequence. In such a case, a complete gene X is present, but due to the lack of an active promoter, no transcript and, thus, no protein is produced. Since this is always a mutation at the front (5′) end of gene X, a scheme such as depicted in FIG. 1B applies.

Furthermore, it is noted that in the case a final recombinant with a mutant gene X is not a problem, for example, in a laboratory strain, a method according to the invention can also be performed as illustrated in FIG. 3. In this example, the mutation is present in the truncated gene X. This method is advantageous when it is difficult or impossible to obtain a strain that comprises a mutated gene X on the genome.

In a preferred embodiment, the invention provides a method for obtaining site-specific, marker-less integration of a sequence of interest in the genome of a cell as outlined herein, wherein the cell is a eukaryotic cell, for example, a plant cell. The plant cell can, for example, be obtained by using the characteristics of a mutated and wild-type erp1 gene.

It is clear that a method according to the invention can be applied both to a prokaryotic cell and to a eukaryotic cell. Typical examples of a cell in which integration according to a method of the invention can be obtained are actinomycetes, more preferably streptomycetes. Production of a protein encoded by the sequence of interest in a prokaryotic cell typically involves secretion of the protein into the extracellular media and, hence, the presence of a marker gene does not interfere significantly with the isolation of marker-free protein. However, in the case a protein is produced in, for example, the leaves of a plant, isolation of the protein can be contaminated with a protein encoded by a marker gene. Furthermore, there is the risk of spread of the marker into the environment when recombinant plants are grown in fields. This is currently one of the biggest problems in plant biotechnology. Acceptance by governments, as well as by the public, would greatly benefit from a method that produces recombinant plants not polluted with additional marker genes. In an even more preferred situation, if only plant sequences are used and, preferably, homologous plant sequences, the recombinant plant will contain only endogenous sequences, lacking DNA from, for example, bacterial or fungal origin. Hence, the present invention is particularly advantageous for providing a eukaryotic cell with a sequence encoding a protein or RNA molecule of interest.

Besides a method for obtaining site-specific, marker-less integration of a sequence of interest in the genome of a cell, the invention also provides a cell obtainable according to any one of the invention's methods. Preferably, the cell is a eukaryotic cell and, even more preferably, the eukaryotic cell is a plant cell. Non-limiting examples of dicot plants are Brassica, potato, tomato, soy bean, sugar beet, and Arabidopsis, and examples of monocot plants are rice, maize, wheat, and barley.

The invention also provides an organism which comprises a cell according to the invention. Preferably, the organism is a non-human organism/animal and, even more preferably, the organism is a plant.

In yet another embodiment, the invention provides a method for producing an antibiotic or a useful protein comprising culturing a cell according to the invention or an organism (preferably a non-human organism/animal) according to the invention and harvesting the antibiotic or protein from the cell, organism or culture.

The invention will be explained in more detail in the following description, which is not limiting the invention.

### DESCRIPTION OF THE FIGURES

FIGS. 1A and 1B. Scheme for marker-less integration into the genome with positive selection criteria.

FIG. 1A. Selection on the basis of a mutation (or small deletion) in gene X, located towards the end of the gene. In this particular case, gene X is represented by glkA, encoding glucose kinase, and sequence Y is represented by the sequence downstream of glkA. Crosses indicate possible regions for homologous recombination, resulting in either situation (1) recombination upstream of cloned DNA or (2) recombination downstream of cloned DNA. Cloned DNA refers to the DNA that needs to be inserted into the host genome. Mutant genes are labeled with an asterisk and the approximate site of mutation, by a dot. Arrows indicate selection or screening steps. Possible (but less likely) recombination events between the mutation in gene X and the cloned DNA are illustrated in FIG. 5. The figure is not drawn to scale.

FIG. 1B. Same as FIG. 1A, but now with mutation in gene X located towards the start of the gene. In this case, gene X is preceded by sequence Y and the sequence of interest. For further details see legend to FIG. 1A.

FIG. 2. Same as FIG. 1A, but now with a gene X that is sensitive to a certain component or condition Z. In such a case, the first recombination step cannot be positively selected. Alternative with mutation towards the start of gene X not shown (for explanation of the difference, see FIGS. 1A and 1B).

FIG. 3. Method starting with mutant gene X on the plasmid. This results in a mutant gene X on the genome. For a more detailed explanation, see FIGS. 1A and 1B. Alternative with mutation towards the start of gene X not shown (for explanation of the difference, see FIGS. 1A and 1B).

FIG. 4. As shown in previous figures, but now introducing a deletion rather than an insertion. The deleted region is for illustration purposes presented as a gene B with flanking sequences, but could also be a stretch of noncoding DNA or otherwise. FIG. 4A, mutation towards end of gene X; FIG. 4B, mutation towards start of gene X. For more detailed explanation, see FIGS. 1A and 1B.

FIG. 5. Possible recombination events between the mutation in gene X and the cloned DNA. A and B refer to possible areas of recombination upstream of the cloned DNA sequence. Recombination through area A is illustrated in FIGS. 1 through 4. Recombination through area B, which may sometimes arise, results in a situation 1B. Prior to continuation of the recombination procedure, this event needs to be excluded by a method such as PCR analysis. This event was not observed in an experiment, where the ratio between the lengths of A and B was 5:2 (see experimental section). For a more detailed explanation, see FIGS. 1A and 1B.

FIG. 6. Sequence of the glkA region amplified from the S. coelicolor M145 genome. Nucleotide numbering refers to the translational start of glkA (the first 12 codons were omitted from the clone to ensure inactivity of the plasmid-borne gene). The DNA was amplified using oligonucleotides glkX (identical to nucleotide positions 37-57) and glkY (complementary to nucleotide positions 2096-2116). These oligonucleotides were designed so as to introduce SmaI and KpnI sites upstream of nt position 37 and downstream of nt position 2116, respectively. Start and stop codons for glkA (SCO2126; stop at 959), ORF6E10.19 (SCO2125; start at 1099, stop at 1860), and ORF6E10.18 (SCO2124; reversed, stop at 1885), are underlined and italicized. The BclI site around nt position 1085 used for cloning is underlined and in bold face. Nucleotide sequence was determined by the Sanger genome sequencing project (Bentley et al. 2002).

FIG. 7. Map of pMBS011. Sequence between NdeI and KpnI sites (clockwise) is derived from pIJ2581. Unique restriction sites shown in bold face. Genes: tsr, thiostrepton-resistance gene (Kieser et al. 2000); bla, β-lactamase gene; lacZ, inactive part of lacZ fragment; F1(+), ori for ssDNA; colE1, E. coli ori (high copy number). Truncated glkA and downstream-located ORF 6E10.19 constitute homologous sequences for recombination (see text).

FIG. 8. Alignment of glucokinases from various microorganisms. Black-shaded residues indicate conserved amino acids, grey-shaded residues indicate conserved similarities. Sequences A-H show highly conserved regions. Several mutations in the conserved boxes A (putative ATP-binding domain), B (putative sugar-binding domain), E, F, and G rendered the glucose kinase from S. coelicolor inactive. Abbreviations of strains from which glucokinases were derived: Sliv, Streptomyces lividans; Scoe, Steptomyces coelicolor; Sxyl, Staphilococcus xylosus; Bsub, Bacillus subtilis; Tmar, Thermatoga maritima; Syne, Synechocystius species; Drad, Deinococcus radians. Glk2 refers to a homologue of glucose kinase in Streptomyces coelicolor, which is the most likely candidate of constituting the secondary glucose kinase activity, which is sometimes induced after prolonged exposure of glkA mutants to MM containing glucose (Angell et al. 1994). N-terminal extensions of S. coelicolor Glk2 and of D. radians Glk not shown.

FIG. 9. Alignment of ETR1 homologues from plants. The four homologues compared are derived from ARA_TH, Arabidopsis thaliana (thale cress; genbank accession P49333), NIC_TA, Nicotiana tabacum (tobacco; GenBank accession 048929), CUC_ME, Cucumis melo (muskmelon; GenBank accession 082436), and LYC_ES, Lycopersicon esculentum (tomato; GenBank accession Q41342). Amino acids mutations resulting in ethylene insensitivity are shown below the sequence. Specific mutations studied were A31V, 162F, C65Y, C65S, A102T.

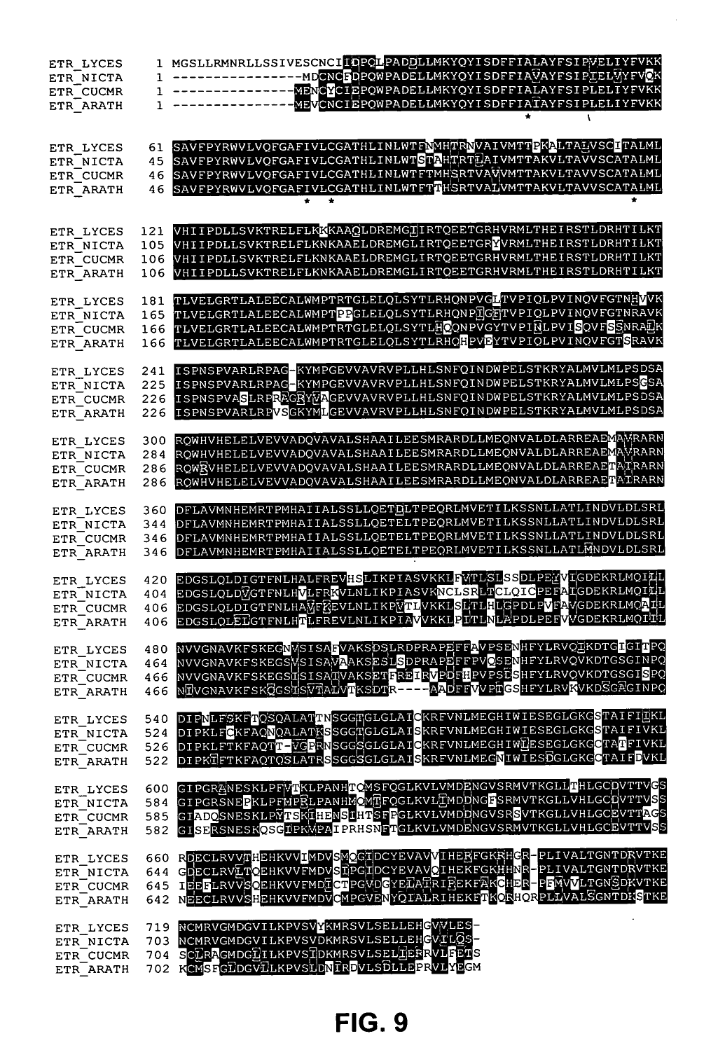

### DETAILED DESCRIPTION OF THE INVENTION

Experimental Part & Results

Bacterial Strains and Culturing Conditions

E. coli K-12 strains JM109 was used for propagating plasmids and was grown and transformed by standard procedures (Sambrook et al. 1989). E. coli ET12567 (MacNeil et al. 1992) was used to isolate DNA for transformation of plasmid DNA to Streptomyces coelicolor. Transformants were selected in L broth containing 1% (w/v) glucose, and ampicillin at a final concentration of 200 μg ml⁻¹. L broth with 1% glucose and 30 μg ml⁻¹ chloramphenicol was used to grow ET12567.

Streptomyces coelicolor A3(2) M145 was obtained from the John Innes Centre strain collection. Protoplast preparation and transformation were performed as described by Kieser et al. (2000). SFM medium (mannitol, 20 g 1⁻¹; soya flour, 20 g 1⁻¹; agar, 20 g 1⁻¹, dissolved in tap water) was used to make spore suspensions. Minimal Medium (MM) and R2YE agar plates (Kieser et al. 2000) were used for selection experiments; R2YE was also used for regenerating protoplasts and, after addition of the appropriate antibiotic, for selecting recombinants. For standard cultivation of Streptomyces, YEME (Kieser et al. 2000) or tryptone soy broth (Difco) containing 10% (w/v) sucrose (designated TSBS) were used. Liquid cultures to select for glucose utilization were performed in NMMP (minimal medium), with 1% (w/v) mannitol or 1% (w/v) glucose as the carbon source.

Construction of pMBS011

As the basis for a recombination plasmid, we used pIJ2581 (5192 bp; Genbank Accession X98363; van Wezel and Bibb, 1996), a construct based on pBluescript SK+ (Strategene), with bla as selectable marker in E. coli, and tsr as selectable marker in Streptomyces. The plasmid has both ColE1 and f1 (+) origins of replication, the latter allowing the production of single-stranded DNA in the presence of helper phage (Sambrook et al. 1989). Single-stranded DNA increases the transformation efficiency in Streptomyces (Hilleman et al. 1991). The plasmid lacks a Streptomyces origin of replication and can, therefore, only be maintained by integration into the host genome through cloned homologous sequences.

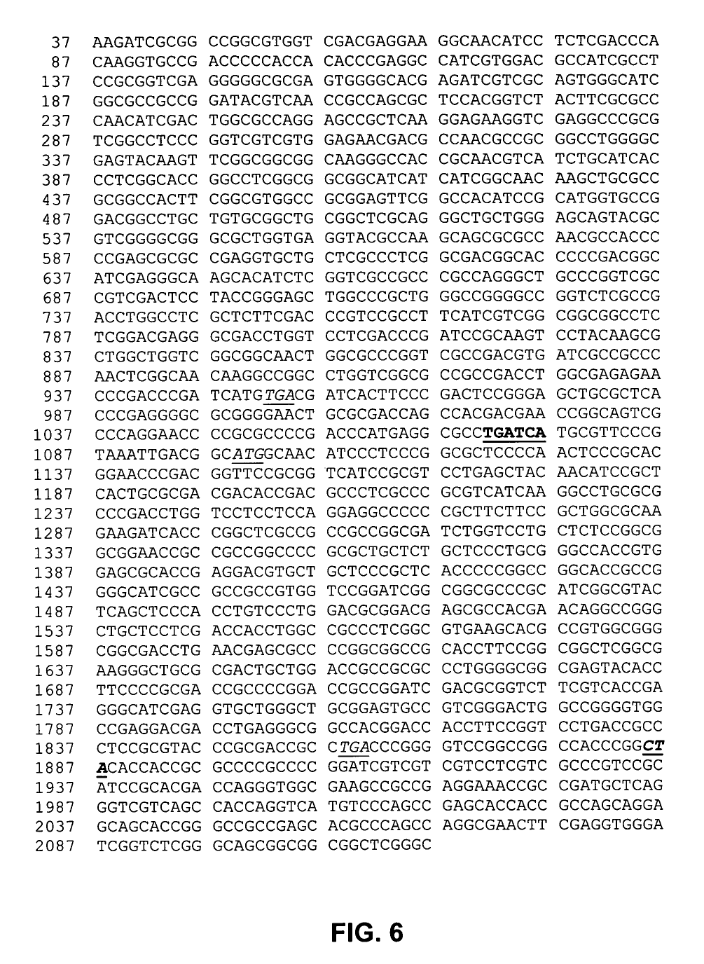

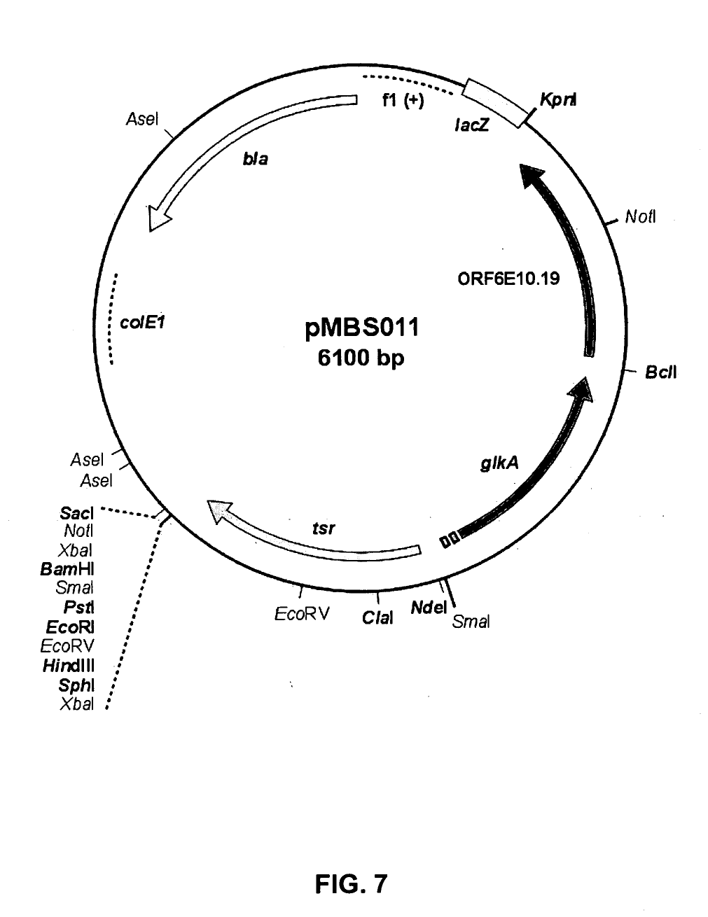

A 2080 bp sequence harboring all but the first 36 bp of glkA, as well as 1162 bp of the downstream sequence, was amplified from the S. coelicolor M145 genome using the 30-mer oligonucleotides glkX and glkY. These oligonucleotides were designed so as to add SmaI and KpnI sites to the beginning and the end of the DNA fragment, respectively. The exact sequence inserted is shown in FIG. 6. This PCR fragment was subsequently cloned into pIJ2581, digested with KpnI and partially digested with SmaI, effectively removing the approximately 1150 bp glkA gene. The resulting construct pMBS011 is shown in FIG. 7. The unique BclI site in pMBS011 is compatible with BamHI and BglII restriction sites and can, for example, be used for cloning inserts from pIJ2925, which is a derivative of pUC19, carrying BglII restriction sites flanking the multiple cloning site (Janssen and Bibb, 1993).

Mutations that Inactivate Glucose Kinase and Confer 2-Deoxy-Glucose Resistance

The non-utilizable glucose analogue 2-deoxy-glucose (2-DOG) is lethal when introduced in bacterial strains that have an active glucokinase (designated glucose kinase in streptomycetes). Strains harboring mutant glkA genes fail to grow on glucose, but are resistant to 2-DOG. Introduction of an active glucokinase or restoration of the wild-type gene by recombination restores full glycolysis and glucose utilization and renders the cells sensitive to 2-DOG.

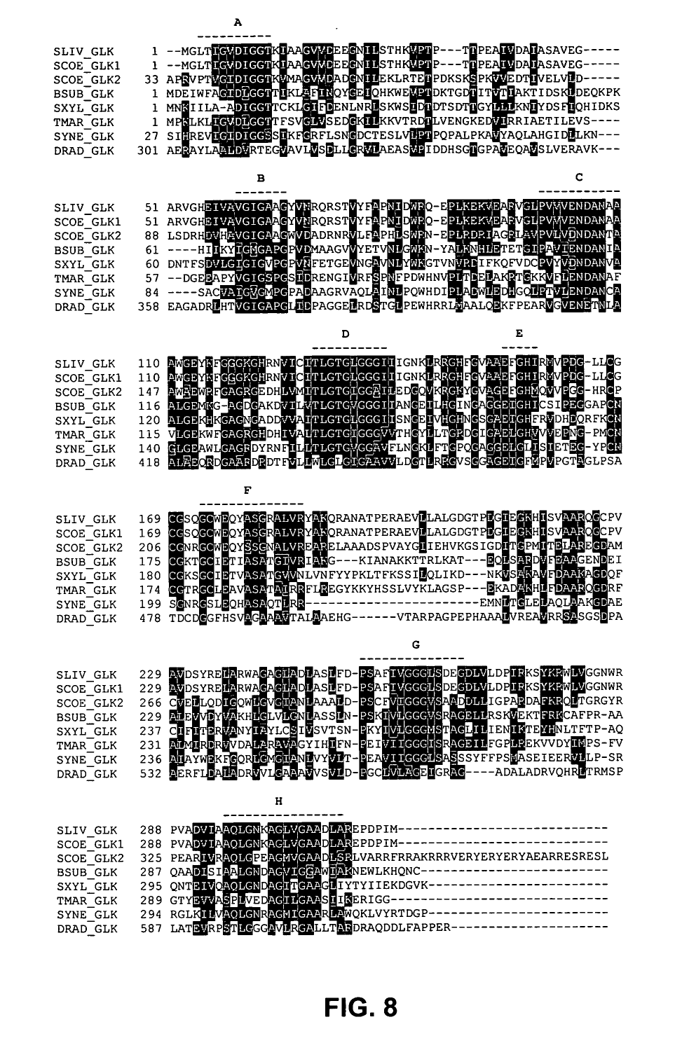

An alignment of several bacterial glucokinases is shown in FIG. 8. Several highly conserved regions can be observed, designated sequences A-H in the figure (overlined). Sequence A represents the P-loop (ATP-binding consensus sequence). Many site-directed mutants have been created in the S. coelicolor glkA gene, resulting in glucose kinases that have lost the ability to phosphorylate glucose. Mutational hotspots, where all mutations made so far result in enzymatic inactivity are, for example, sequences A, E and G.

To create Streptomyces coelicolor strains mutant for glucose kinase, these were grown on solid MMD plates, consisting of MM (Kieser et al. 2000) with 1% (w/v) mannitol and 100 mM 2-deoxyglucose, the latter compound being lethal for Glk⁺ strains. Therefore, colonies that develop on this medium have to be Glk⁻. Colonies that were able to grow on MMD were selected and tested for glucose kinase activity. Glucose kinase-deficient (ΔglkA) strains were checked by PCR, which showed that the nature of the mutations varied from large deletions to point mutations. For the experiments described herein, a generated mutant glkA harboring a small deletion corresponding to aa 257-262 (see FIG. 8) was used.

Marker-Less Insertion of the EGFP Gene into the S. coelicolor Genome with Positive Selection Construction of the Insertion Vector

For demonstration of the integration method, using pMBS011 as integration vector, the gene for EGFP (enhanced green fluorescent protein) was chosen to be inserted into the S. coelicolor genome. For this purpose, we amplified an approximately 1 kb DNA fragment harboring the EGFP gene and its RBS with oligonucleotides GfpX and GfpY, designed so as to provide BglII sites at either end. The PCR-amplified EGFP gene-containing DNA fragment was digested with BglII and inserted into BclI-digested pMBS011. DNA from the latter was isolated from E. coli strain ET12567 (mutant for several modification genes, including dam dcm; McNeill et al. 1992) to allow digestion of the normally dam-methylated BclI site. After ligation, the DNA was re-digested with BclI so as to guarantee the absence of vector without insert. All colonies tested contained the expected plasmid, which was designated pMBS012.

Construct pMBS012 has the N-truncated but otherwise wild-type glkA gene followed by the EGFP gene, which, in turn, is followed by ORFs 6E10.19 and the end of ORF6E10.18, which is oppositely oriented (not indicated in FIG. 7). This construct allows integration of the EGFP gene behind glkA on the S. coelicolor genome. The resulting recombinant genome should preferably harbor no heterologous sequences (other than the desired 800 bp EGFP gene flanked by the fused BclI-BglII sites).

Insertion of the EGFP Gene into the S. coelicolor Genome

An S. coelicolor glkA mutant lacking the codons for amino acid residues 257-262 (IVGGGL, FIG. 8) was transformed with pMBS012 and colonies were selected for resistance to thiostrepton. Subsequently, recombinants were plated on MM plates with mannitol as the sole carbon source (Kieser et al.) and containing 100 mM 2-deoxy glucose and 10 μg/ml thiostrepton.

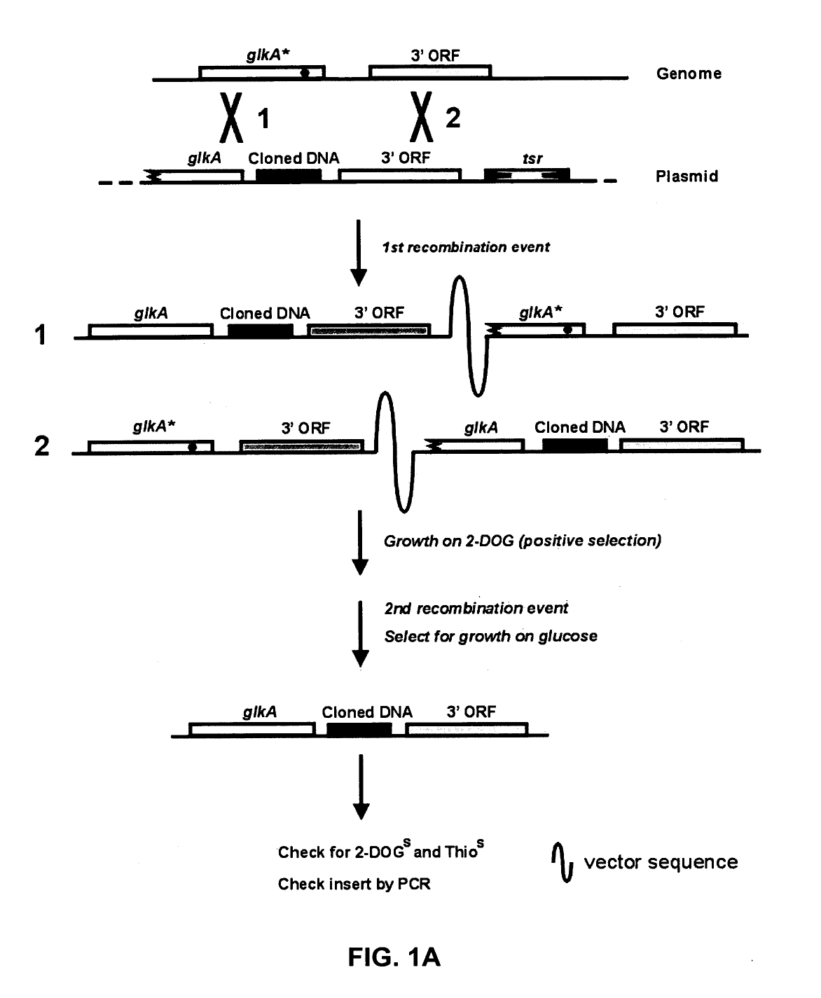

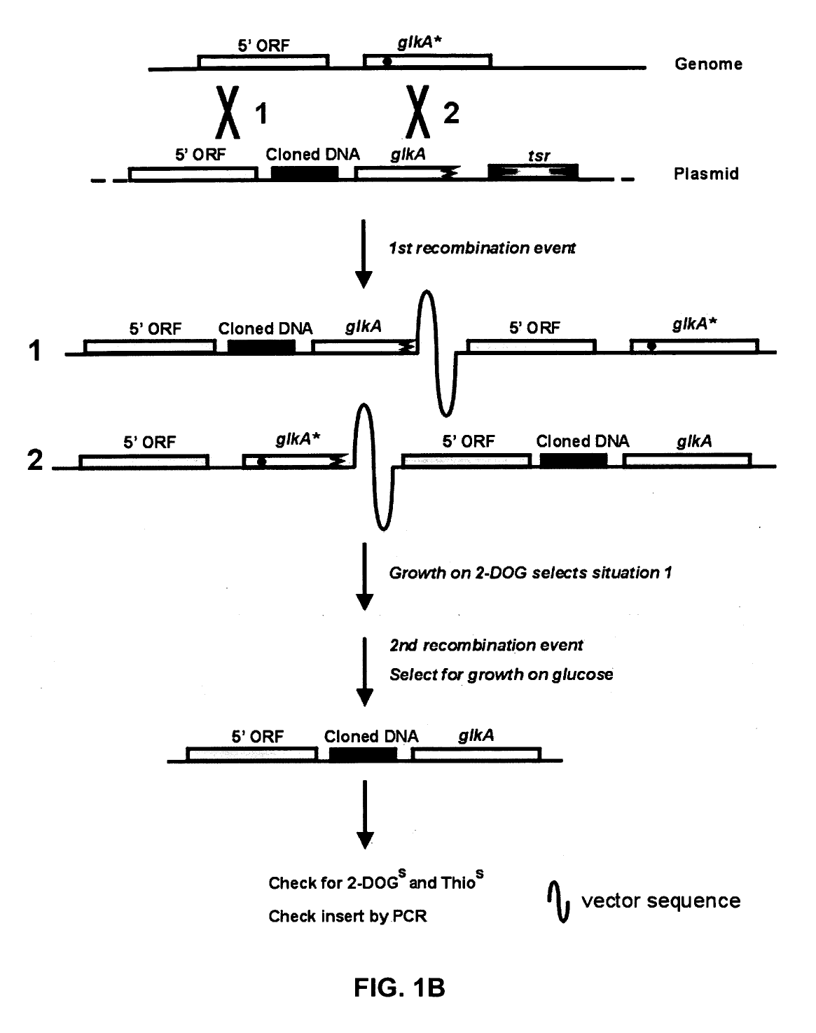

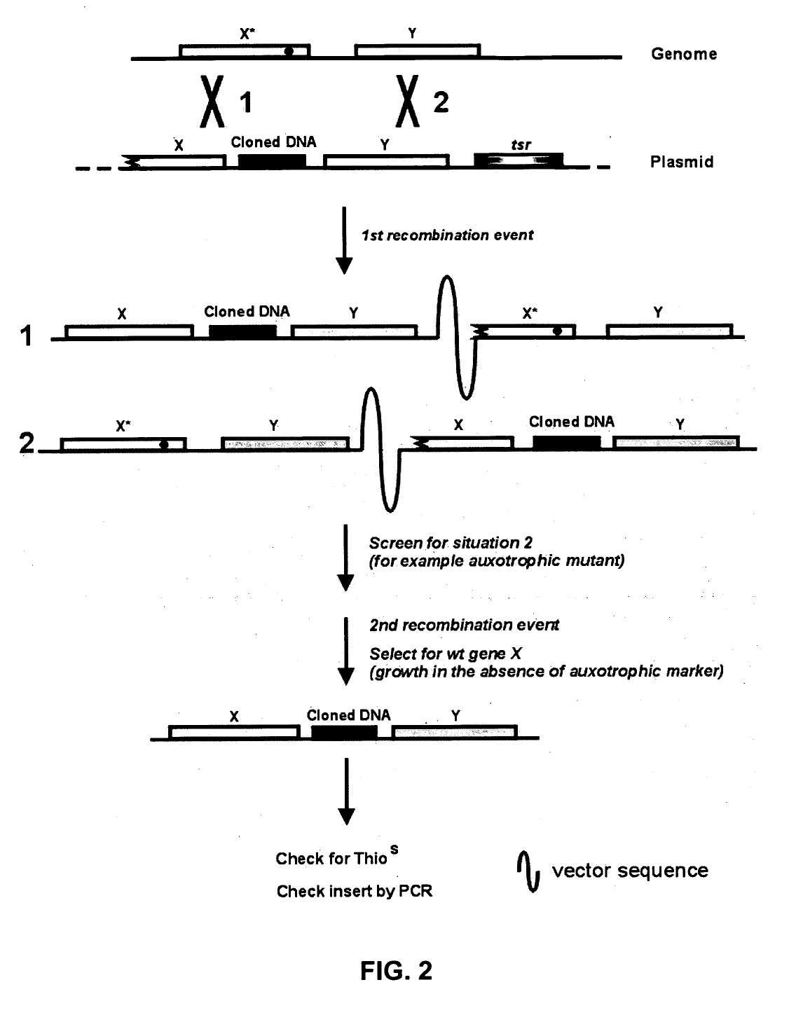

In this way, positive selection was achieved of recombinants in which recombination event 2 (FIG. 1A) has occurred through recombination in sequence Y (i.e., downstream of the EGFP gene). This results in a complete but catalytically inactive mutant glucose kinase and a truncated wild-type copy, rendering the recombinant 2-DOG resistant. The other type of recombination (event 1 in FIG. 1A), results in recombinants with a wild-type and catalytically active glkA gene, which, therefore, fail to grow on 2-DOG. Three colonies were checked and found to have undergone the correct recombination event.

The viable primary recombinants were streaked on MM plates with 2-DOG and subsequently replicated onto MM with glucose as the sole carbon source to allow recombination events to occur and spores harvested. These were used to inoculate a liquid NMMP culture (Kieser et al. 2000) with mannitol as the sole carbon source, grown until OD₆₀₀ of 0.5, washed twice in NMMP without carbon source, resuspended in NMMP with glucose as the sole carbon source, and grown until stationary phase was reached (typically overnight). Only mycelium with a wild-type glucose kinase gene can utilize glucose and such recombinants must have arisen from a further recombination event through the homologous glkA sequences, resulting in a wild-type glucose kinase gene followed by the EGFP gene on the genome.

The mycelium was plated on MM plates with glucose as the sole carbon source to select the population with a wild-type glkA gene. Colonies that appeared were tested for sensitivity to 2-DOG and thiostrepton. The majority of the colonies tested harbored a wild-type glkA gene and were sensitive to thiostrepton.

Southern hybridization on genomic DNA isolated from two independent colonies confirmed that these had the expected EGFP insertion.

Thus, we succeeded in inserting a DNA fragment into the genome of S. coelicolor M145, without leaving any selectable marker or other undesired sequences behind, and with both recombination steps positively selectable, namely resistance to thiostrepton and 2-DOG (step 1), and ability to grow on glucose (step 2). DNA sequencing confirmed the presence of the expected insert.

Thus, we believe that this is an important step forward in creating recombinant microorganisms, especially those which are notoriously hard to screen, such as actinomycetes.

Solving Possible Polar Effects of Insertions on the Transcription of Downstream Genes

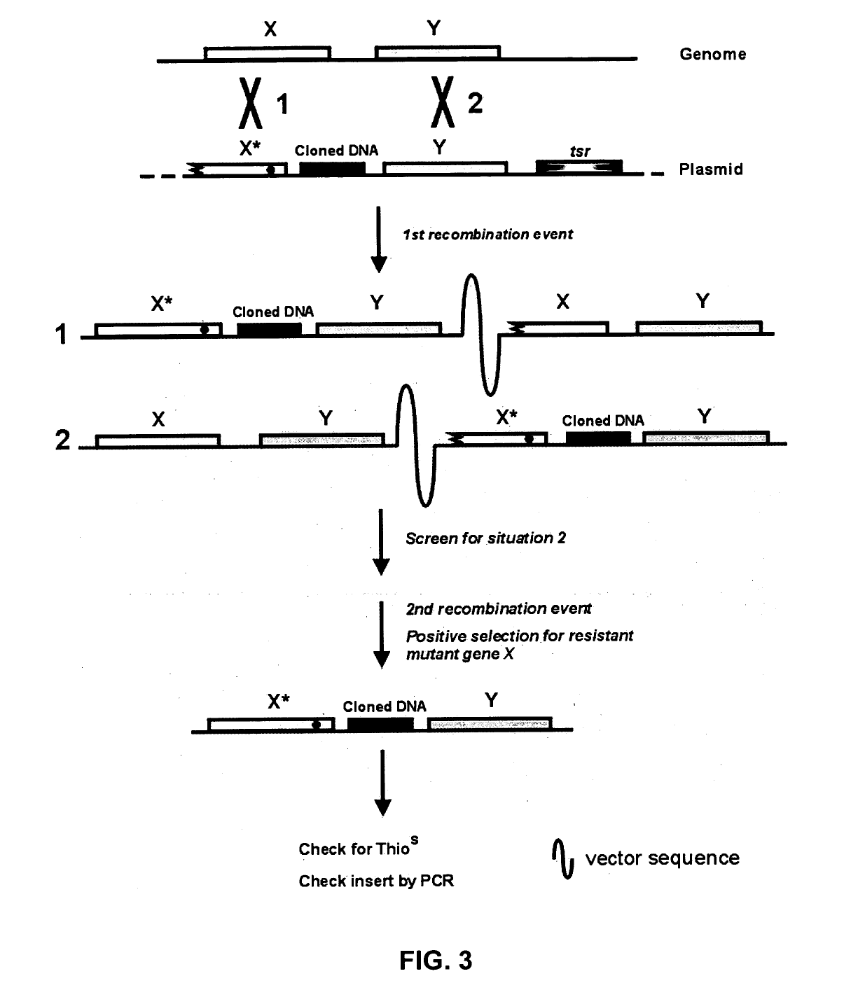

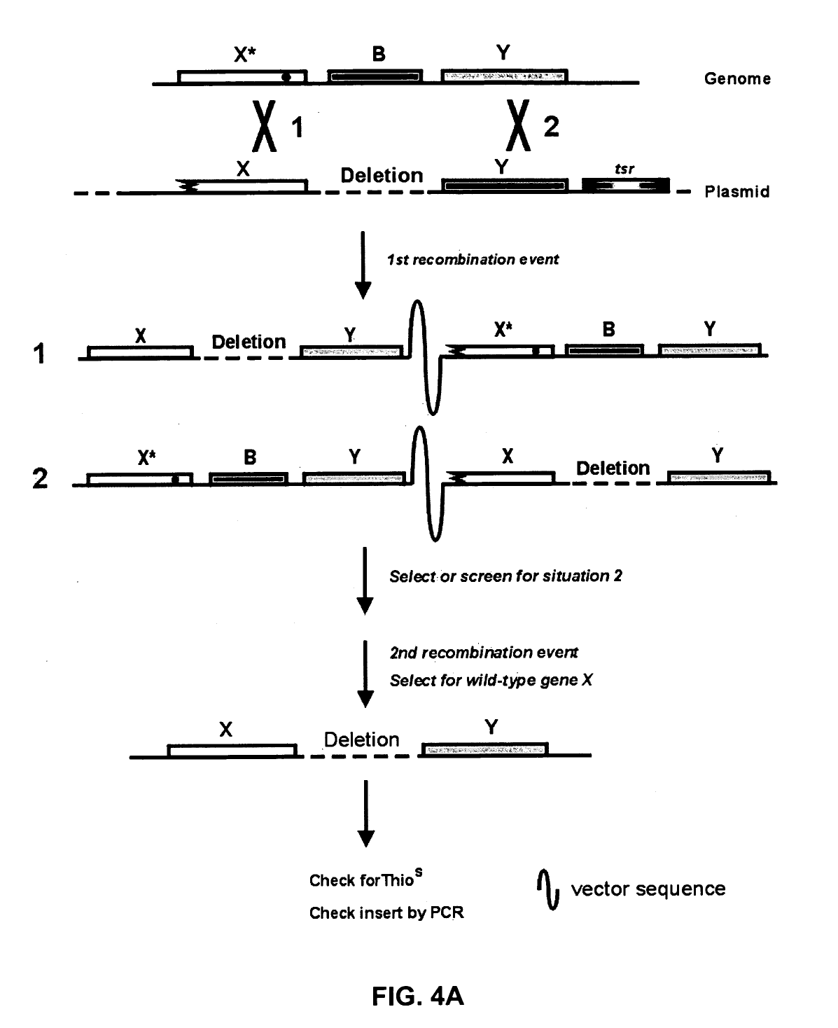

It is possible that insertion of a DNA sequence into the genome affects the transcription of downstream-located genes, resulting in so-called polar effects. For example, this occurs if, in the final situation in FIGS. 1A, 2 and 3, the inserted DNA alters and/or blocks transcription of genes in sequence Y and/or downstream of it; similarly, in FIG. 1B, insertion of DNA could affect transcription of gene X (glkA) and possibly also of downstream-located genes. In such a case, it is desirable or, in the case of genes indispensable for growth or selection, essential to provide promoter sequences immediately 3′ of the inserted DNA on the disruption construct to ensure proper transcription of downstream genes.

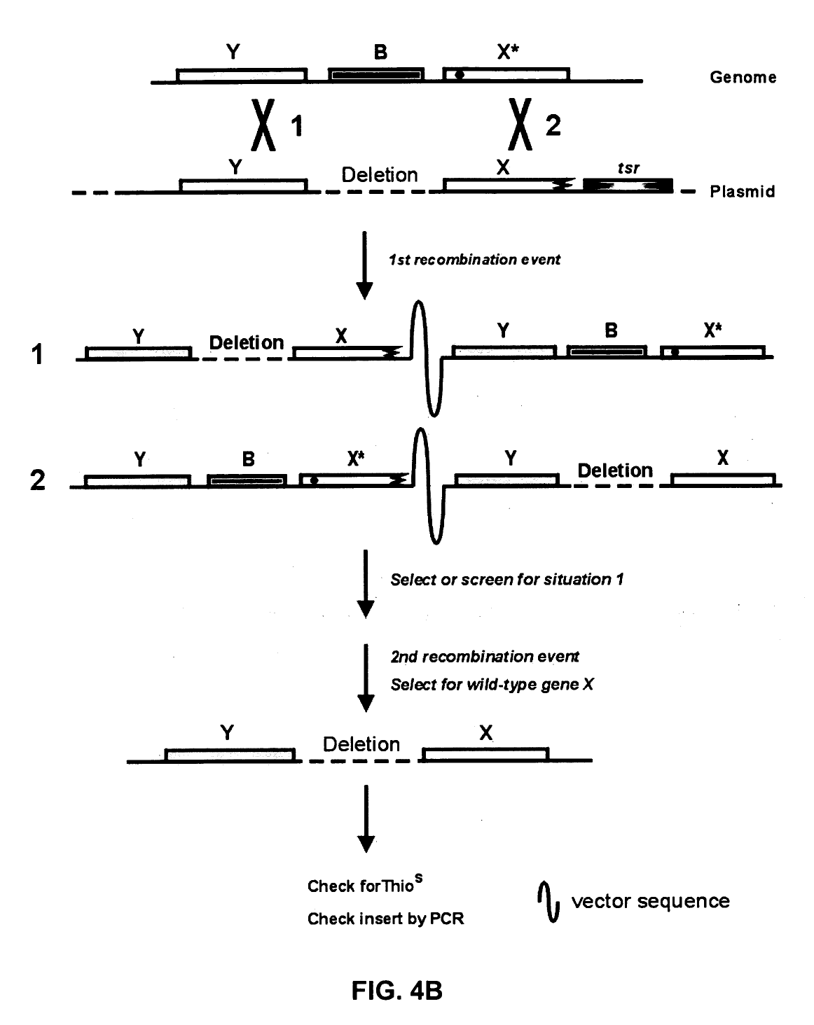

In a more specific case, gene X and sequence Y are also part of the operon, where on the genome they are immediately preceded by the operon promoter and followed by one or more genes that also depend on this promoter. In such a case, insertion of a plasmid by recombination through gene X or sequence Y results in block of transcription of all downstream-located genes. This is lethal if one or more of the downstream-located genes is essential for growth. Negative effects of the insertion can only be counteracted by making sure that two promoters are present on the plasmid, one promoter either upstream of the truncated gene X (FIGS. 1A, 2, 3, and 4A) or upstream of sequence Y (FIGS. 1B and 4B), and a second promoter, either between the cloned DNA and the truncated gene X (FIGS. 1B and 4B) or between the inserted DNA and sequence Y (FIGS. 1A, 2, 3, and 4A).

In the case of a glucose kinase gene, it is likely that insertion of DNA into the BclI site (FIG. 6) will block transcription of the downstream-located ORF6E10.19. However, from earlier experiments (Kelemen et al. 1995), it is known that deletion of this gene does not affect growth or morphology. This was confirmed by the wild-type phenotype of the final recombinant harboring the EGFP gene between glkA and ORF6E10.19 on the genome.

Recombination Events Between Mutation in Gene X and the Cloned DNA

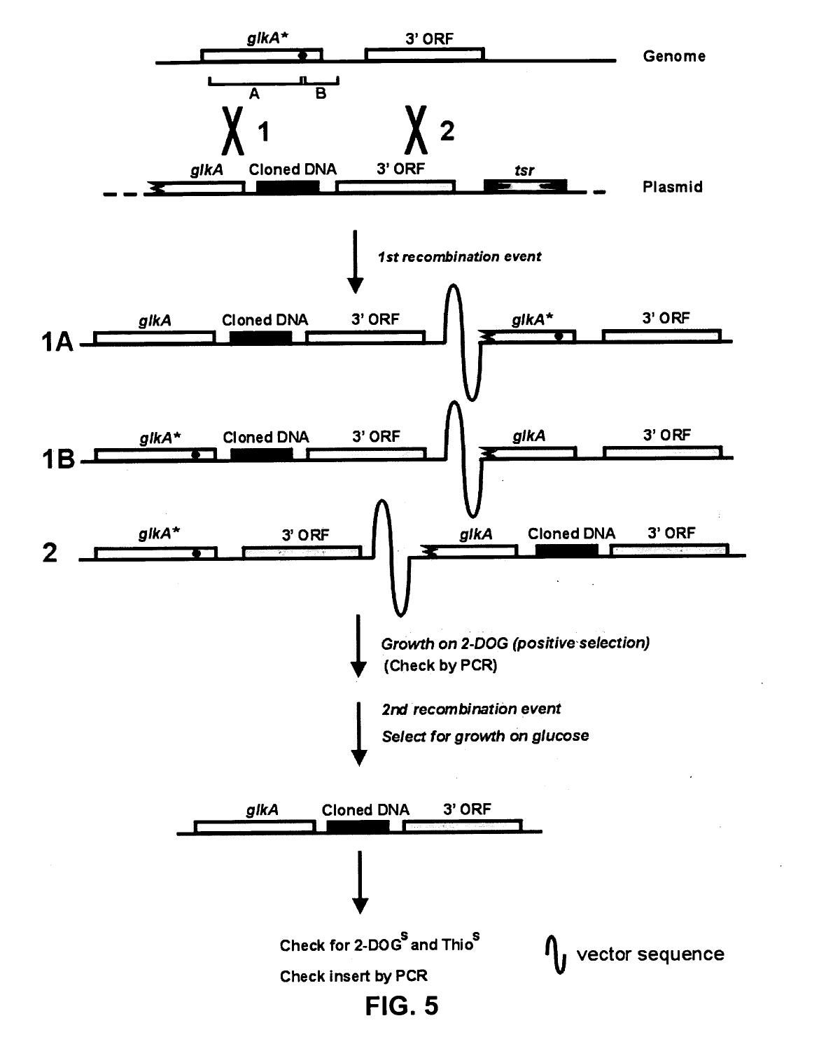

There is a possibility of a recombination event upstream of the cloned DNA but downstream of the site of mutation. This event is exemplified in FIG. 5 and depicted as 1B. Before proceeding with the second recombination step, this possibility needs to be ruled out, for example, by PCR of genomic DNA of a few recombinants.

The chance P_{B} for this undesirable recombination to occur can, in our experience, be estimated by the formula: P_{B}=½(A/B)²×100%. For example, with a ratio A:B=2:1, it follows that the chance of recombination through sequence B is approximately 13%. In most cases, the experimenter will be able to choose the situation such that the ratio A:B is much larger, as in the cases listed in Table 2, so that P_{B} becomes negligible. While in principle area B should be minimized, in practice, it follows that as long as the ratio A:B exceeds 2:1, checking a few recombinants is sufficient to identify the correct recombinant to enter recombination step 2.

In the glkA experiment described in the experimental section where the ratio A:B is around 5:2, all three colonies checked had undergone recombination through area A.

In the example of the use of the EPR1 gene for recombination in plants, mutations all lie between nucleotide positions 100-300, while the whole gene is more than 2000 bp long. In such a case, the chance of finding the desired recombination through area A is close to 100%.

TABLE 1
Overview of selection criteria in recombination schemes in FIGS. 1-4.

| | | | Insertion or | | 1^{st} step | | 2^{nd} step | |
|---|---|---|---|---|---|---|---|---|
| Figure | Gene X | Organism | deletion | Z (step 1) | selectable | Z (step 2) | selectable | Wt |
| 1 | Gene sensitive AND essential | Bacterium | Insertion | 2-DOG | Y | Glucose | Y | Y |
| | Example: glkA | | | | | | | |
| | Gene sensitive AND essential | Bacterium | Insertion | Streptomycin | Y | Streptomycin | Y | Y |
| | Example: streptomycin | | | | | | | |
| | dependent rpsL | | | | | | | |
| 2 | Peroxidase, catalase gene | Bacterium, | Insertion | Peroxide | N | Peroxide | Y | Y |
| | | Fungus, plant cell | | | | | | |
| | Gene that can be made Ts | | Insertion | High Temp. | N | High Temp. | Y | Y |
| | Examples: fts (cell division) | | | | | | | |
| | genes, genes for translation | | | | | | | |
| | factors. | | | | | | | |
| | Biosynthesis gene (mutation | Bacterium, | Insertion | Lack of aux. | N | Lack of aux. | Y | Y |
| | gives auxotrophy) | Fungus, Plant cell | | marker | | marker | | |
| | Examples: amino acid, | | | | | | | |
| | vitamin, or nucleotide | | | | | | | |
| | biosynthesis genes | | | | | | | |
| 3 | Gene sensitive to compound | Bacterium, | Insertion | Antibiotic | N | Antibiotic | Y | N |
| | such as an antibiotic; | Fungus, Plant cell | | | | | | |
| | Mutation on the plasmid! | | | | | | | |
| | Examples: genes for r-proteins, | | | | | | | |
| | rRNA, RNA polymerase | | | | | | | |
| | subunit, DNA synthesis | | | | | | | |
| | machinery, translation factors | | | | | | | |
| 4 | As in 1-3 | — | Deletion | See 1-3 | N | — | Y | Y |

“Selectable” means positive selection for desired recombination event possible. Non-selectable means desired situation needs to be screened for, e.g., by replicating colonies to agar plates with and without the selectable compound or condition Z, looking for Z-sensitive colonies. Situation 3 differs from 1 and 2 in that the mutation needed for screening/selection lies on the plasmid rather than on the genome.

TABLE 2
Candidate selection genes (gene X)

| Gene product, gene | Organism | Residue number | Mutation | Phenotype | Applicable to |
|---|---|---|---|---|---|
| Glucose kinase, glkA | Streptomyces coelicolor | | G11V | 2-DOG resistant, no glucose utilization | prokaryotes |
| | | | K13V | | |
| | | | G260V | | |
| | | | C263A | | |
| r-protein S12, rpsL | Escherichia coli | 124 | P90L | streptomycin dependent | prokaryotes |
| | | | P90R | | |
| | | | K42Q | | |
| | Streptomyces coelicolor | 123 | K43Q | | |
| Catalase, katG | Escherichia coli | 726 | G119D | sensitive to H2O2 stress | prokaryotes |
| | Synechocystis PCC 6803 | 754 | H123E | | |
| | | | H123Q | | |
| Catalase, cpeB | Streptomyces coelicolor | 740 | H109E | | |
| | | | H109Q | | |
| EF-Tu, tuf | Escherichia coli | 394 | A375T | kirromycin resistant | prokaryotes |
| | Streptomyces coelicolor | 397 | A378T | | |
| EPR1 | Arabidopsis, Nicotiana | 738 | A31V | ethylene sensitive | plants |
| | | | I62F | | |
| | | | C65Y | | |
| | | | C65S | | |
| | | | A102T | | |
| r-protein L11, rplK | Halobacterium | 163 | P18S or P18T | thiostrepton resistant | prokaryotes |
| | Escherichia coli | 142 | P22S or P22T | | |
| 23S rRNA, rrn | Escherichia coli | 2903 | A2058G | macrolide resistant | prokaryotes |
| | | | A2059G | | |
| 16S rRNA, rrn | Escherichia coli | 1541 | U1192 | spectinomycin resistant | prokaryotes |

### REFERENCES

Angell S., C. G. Lewis, M. J. Buttner and M. J. Bibb (1994) Glucose repression in Streptomyces coelicolor A3(2): a likely regulatory role for glucose kinase. Mol. Gen. Genet. 244:135-143.
Angell S., E. Schwarz and M. J. Bibb (1992) The glucose kinase gene of Streptomyces coelicolor A3(2): its nucleotide sequence, transcriptional analysis and role in glucose repression. Mol. Microbiol. 6:2833-2844.
Bentley S. D., K. F. Chater, A. M. Cerdeno-Tarraga, G. L. Challis, N. R. Thomson, K. D. James, D. E. Harris, M. A. Quail, H. Kieser, D. Harper, A. Bateman, S. Brown, G. Chandra, C. W. Chen, M. Collins, A. Cronin, A. Fraser, A. Goble, J. Hidalgo, T. Hornsby, S. Howarth, C. H. Huang, T. Kieser, L. Larke, L. Murphy, K. Oliver, S. O'Neil, E. Rabbinowitsch, M. A. Rajandream, K. Rutherford, S. Rutter, K. Seeger, D. Saunders, S. Sharp, R. Squares, S. Squares, K. Taylor, T. Warren, A. Wietzorrek, J. Woodward, B. G. Barrell, J. Parkhill and D. A. Hopwood (2002) Complete genome sequence of the model actinomycete Streptomyces coelicolor A3(2). Nature 417:141-147.
Bleecker A. B., M. A. Estelle, C. Somerville and H. Kende (1988) Insensitivity to ethylene conferred by a dominant mutation in Arabidopsus thaliana. Science 241:1086-1089.
Buttner M. J., K. F. Chater M. J. and Bibb (1990) Cloning, disruption, and transcriptional analysis of three RNA polymerase sigma factor genes of Streptomyces coelicolor A3(2). J. Bacteriol. 172:3367-3378.
Janssen G. R. and M. J. Bibb (1993) Derivatives of pUC18 that have BglII sites flanking a modified multiple cloning site and that retain the ability to identify recombinant clones by visual screening of Escherichia coli colonies. Gene 124:133-134.
Kelemen G. H., K. A. Plaskitt, C. G. Lewis, K. C. Findlay and M. J. Buttner (1995) Deletion of DNA lying closes to the glkA locus induces ectopic sporulation in Streptomyces coelicolor A3(2). Mol. Microbiol. 17:221-230.
Kieser T., M. J. Bibb, M. J. Buttner, K. F. Chater and D. A. Hopwood (2000) Practical Streptomyces genetics. Norwich, U.K.: John Innes Foundation.
Knoester M., L. C. van Loon, J. van den Heuvel, J. Hennig, J. F. Bol and H. J. M. Linthorst (1998) Ethylene-insensitive tobacco lacks nonhost resistance against soil-borne fingi. Proc. Natl. Acad. Sci. 95:1933-1937.
MacNeil D. J., K. M. Gewain, C. L. Ruby, G. Dezeny, P. H. Gibbons and T. MacNeil (1992) Analysis of Streptomyces avermitilis genes required for avermectin biosynthesis utilising a novel integration vector. Gene 111: 1-68.
Sambrook J., E. F. Fritsch and T. Maniatis (1989) Molecular cloning: a laboratory manual. In: 2nd ed. Cold Spring Harbor Laboratory Press, Cold Spring Harbor, N.Y.
Timms A. R., H. Steingrimsdottir, A. R. Lehmann and B. A. Bridges (1992) Mutant sequences in the rpsL gene of Escherichia coli B/r: mechanistic implications for spontaneous and ultraviolet light mutagenesis. Mol. Gen. Genet. 232:89-96.
van Wezel G. P. and M. J. Bibb (1996) A novel plasmid that used the glucose kinase gene (glkA) for the positive selection of stable gene disruptants in Streptomyces. Gene 182:229-230.
van Wezel G. P., E. Takano, E. Vijgenboom, L. Bosch and M. J. Bibb (1995) The tuf3 gene of Streptomyces coelicolor A3(2) encodes an inessential elongation factor Tu that is apparently subject to positive stringent control. Microbiology 141:2519-2528.

1. A method for obtaining site-specific, marker-less integration of a sequence of interest into a cell's genome, wherein said genome comprises a selectable or screenable gene X and a sequence Y, said method comprising:
providing the cell with a plasmid comprising:
a truncated version of gene X,
a substantial part of sequence Y,
a sequence of interest located between said truncated version of gene X and said sequence Y, and
a selection marker located outside the sandwich of said truncated version of gene X, said sequence of interest, and said sequence Y
selecting for a first recombination event by using said selection marker, thereby obtaining a cell in which said plasmid has integrated via homologous recombination into the cell's genome,
selecting or screening for said selectable or screenable gene X, thereby obtaining a cell with a recombinant genome in which recombination has occurred through sequence Y of the genome and sequence Y of the plasmid, and
selecting positively for a second recombination event, thereby obtaining a cell with a recombinant genome in which an internal recombination event has occurred through gene X and said truncated version of gene X.

2. The method according to claim 1, wherein said plasmid essentially cannot replicate during said first recombination event.

3. The method according to claim 1, further comprising:
checking, after the second recombination event, for loss of said selection marker of said plasmid.

4. The method according to claim 1, wherein said obtained cell in which an internal recombination event has occurred through gene X and the truncated version of gene X is checked for the presence of said sequence of interest.

5. The method according to claim 1, wherein said selectable or screenable gene X is selectable or screenable via a component or a chemical and/or physical condition.

6. The method according to claim 1, wherein said cell is dependent on the presence of said component or chemical and/or physical condition due to the presence of said selectable or screenable gene X.

7. The method according to claim 1, wherein said truncated version of gene X is inactive through truncation, but otherwise original.

8. The method according to claim 1, wherein said final recombinant has, except for the desired insertion, an original genome.

9. The method according to claim 1, wherein both recombination steps are selectable.

10. A method for obtaining site-specific, marker-less integration of a sequence of interest into a cell's genome, wherein said genome comprises a gene X which, as a result of a mutation, is essentially insensitive to a certain component or condition Z, said genome further comprising a sequence Y, said method comprising:
providing said cell with a plasmid, which plasmid comprises:
a truncated inactive, but otherwise original, version of gene X,
a substantial part of sequence Y,
a sequence of interest located between said truncated inactive, but otherwise original, version of gene X and said sequence Y, and
a selection marker located outside the sandwich of said truncated inactive, but otherwise original, version of gene X, said sequence of interest and said sequence Y,
selecting for a first recombination event by using said selection marker of said plasmid, thereby obtaining a cell in which said plasmid has integrated via homologous recombination into the cell's genome,
selecting or screening for gene X which is essentially insensitive to a certain component or condition Z, by using component or condition Z, thereby obtaining a cell with a recombinant genome in which recombination has occurred through sequence Y of the genome and sequence Y of the plasmid, and
selecting positively for a second recombination event via component or condition V, thereby obtaining a cell with a recombinant genome in which said second recombination event has occurred internally through the sequences of gene X and said truncated inactive, but otherwise original, version of gene X.

11. The method according to claim 10, wherein said sequence Y of the genome is located downstream of said gene X which, as a result of a mutation, is essentially insensitive to a certain component or condition Z, and wherein said plasmid comprises:
a 5′ truncated inactive, but otherwise original, version of gene X,
a sequence of interest located downstream of said 5′ truncated inactive, but otherwise original, version of gene X,
a substantial part of sequence Y located downstream of said sequence of interest, and
a selection marker located outside the sandwich of said 5′ truncated inactive, but otherwise original, version of gene X, said sequence of interest and said sequence Y,
and wherein said second recombination event has occurred internally through the sequences upstream of the mutation in gene X and said 5′ truncated inactive, but otherwise original, version of gene X.

12. The method according to claim 10, wherein said gene X which, as a result of a mutation, is essentially insensitive to a certain component or condition Z, is mutated close to the 3′ end of said gene X.

13. The method according to claim 10, wherein said mutation in gene X comprises a point mutation.

14. The method according to claim 10, wherein said substantial part of sequence Y is approximately of the same length as the truncated inactive, but otherwise original, version of gene X.

15. The method according to claim 10, wherein said gene X which, as a result of a mutation, is essentially insensitive to a certain component or condition Z is mutated glkA.

16. The method according to claim 15, wherein said mutated glkA is mutated as depicted in FIG. 8.

17. The method according to claim 10, wherein said component or condition Z is 2-deoxy-glucose.

18. The method according to claim 10, wherein said component or condition V is glucose.

19. The method according to claim 10, wherein said gene X which, as a result of a mutation, is essentially insensitive to a certain component or condition Z is mutated rpsL and component or condition Z and component or condition V are both streptomycin.

20. A method for obtaining site-specific, marker-less integration of a sequence of interest into a cell's genome, wherein said genome comprises a mutated gene X and wherein said cell is, due to said mutated gene X, dependent on the presence of a certain component or condition Z, said genome further comprising a sequence Y, said method comprising:
providing said cell with a plasmid, which plasmid comprises:
a truncated inactive, but otherwise original, version of gene X,
a substantial part of sequence Y,
a sequence of interest located between said truncated inactive, but otherwise original, version of gene X and said sequence Y, and
a selection marker located outside the sandwich of said truncated inactive, but otherwise original, version of gene X, said sequence of interest and said sequence Y,
selecting for a first recombination event by using said selection marker of said plasmid, thereby obtaining a cell in which said plasmid has integrated via homologous recombination into the cell's genome,
selecting or screening for a recombinant cell which requires the presence of component or condition Z, thereby obtaining a cell with a recombinant genome in which recombination has occurred through sequence Y of the genome and sequence Y of the plasmid, and
selecting positively for a second recombination event by identifying a recombinant cell which does not require the presence of component or condition Z, thereby obtaining a cell with a recombinant genome in which said second recombination event has occurred internally through the sequences of the gene X and said truncated inactive, but otherwise original, version of gene X.

21. The method according to claim 20 wherein said sequence Y of the genome is located downstream of said mutated gene X and wherein said plasmid comprises:
a 5′ truncated inactive, but otherwise original, version of gene X,
a sequence of interest located downstream of said 5′ truncated inactive, but otherwise original, version of gene X,
a substantial part of sequence Y located downstream of said sequence of interest, and
a selection marker located outside the sandwich of said 5′ truncated inactive, but otherwise original, version of gene X, said sequence of interest and said sequence Y,
and wherein said second recombination event has occurred internally through the sequences upstream of the mutation in gene X and said 5′ truncated inactive, but otherwise original, version of gene X.

22. The method according to claim 20, wherein said mutated gene X is a mutated amino acid biosynthesis gene and component or condition Z is the corresponding amino acid.

23. The method according to claim 20, wherein said mutated gene X is a mutated vitamin biosynthesis gene and component or condition Z is the corresponding vitamin.

24. A method for obtaining site-specific, marker-less integration of a sequence of interest into a cell's genome, wherein said genome comprises a gene X which, as a result of a mutation, is essentially sensitive to a certain component or condition Z, said genome further comprising a sequence Y, said method comprising:
providing said cell with a plasmid which plasmid comprises:
a truncated inactive, but otherwise original, version of gene X,
a substantial part of sequence Y,
a sequence of interest located between said truncated inactive, but otherwise original, version of gene X and said sequence Y, and
a selection marker located outside the sandwich of said truncated inactive, but otherwise original, version of gene X, said sequence of interest and said sequence Y,
selecting for a first recombination event by using said selection marker of said plasmid, thereby obtaining a cell in which said plasmid has integrated via homologous recombination into the cell's genome,
screening for a recombinant cell which is sensitive to a certain component or condition Z, thereby obtaining a cell with a recombinant genome in which recombination has occurred through sequence Y of the genome and sequence Y of the plasmid, and
selecting positively for a second recombination event by identifying a recombinant cell which is insensitive to component or condition Z, thereby obtaining a cell with a recombinant genome in which an internal recombination event has occurred through the sequences of gene X and said truncated inactive, but otherwise original, version of gene X.

25. The method according to claim 24, wherein said sequence Y of the genome is located downstream of said gene X which, as a result of a mutation, is essentially sensitive to a certain component or condition Z and wherein said plasmid comprises:
a 5′ truncated inactive, but otherwise original, version of gene X,
a sequence of interest located downstream of said 5′ truncated inactive, but otherwise original, version of gene X,
a substantial part of sequence Y located downstream of said sequence of interest, and
a selection marker located outside the sandwich of said 5′ truncated inactive, but otherwise original, version of gene X, said sequence of interest and said sequence Y,
and wherein said second recombination event has occurred internally through the sequences upstream of the mutation gene X and said 5′ truncated inactive, but otherwise original, version of gene X.

26. The method according to claim 24, wherein said gene X which, as a result of a mutation, is essentially sensitive to a certain component or condition Z is a mutated peroxide or catalase and component or condition Z is H₂O₂.

27. The method according to claim 24, wherein said gene X which, as a result of a mutation, is essentially sensitive to a certain component or condition Z is a mutated β-lactamase and component or condition Z is a β-lactam-antibiotic or wherein said gene X which, as a result of a mutation, is essentially sensitive to a certain component or condition Z is mutated such that the said cell is thermosensitive and condition Z is a change in temperature.

28. The method according to claim 1, wherein said cell is a eukaryotic cell.

29. The method according to claim 28, wherein said eukaryotic cell is a plant cell.

30. The method according to claim 1, wherein said integration of a sequence of interest in the genome of a cell results in a deletion in said genome.

31. The method according to claim 10, wherein said plasmid essentially cannot replicate during said first recombination event.

32. The method according to claim 10, further comprising:
checking, after the second recombination event, for loss of said selection marker of said plasmid.

33. The method according to claim 10, wherein said obtained cell in which an internal recombination event has occurred through gene X and the truncated version of gene X is checked for the presence of said sequence of interest.

34. A cell obtainable by the method according to claim 1.

35. The cell of claim 34 which is a eukaryotic cell.

36. The cell of claim 34 which is a plant cell.

37. An organism comprising the cell of claim 34.

38. The organism of claim 37 which is a plant.

39. A method for producing an antibiotic or a protein, said method comprising:
culturing the cell of claim 34, and
harvesting said antibiotic or protein from said cell, organism or culture.


SEQUENCE LISTING (13 sequences)

SEQ ID NO: 1 - protein, 6 aa, Artificial
  Deleted part of an S. coelicolor glkA mutant
IVGGGL

SEQ ID NO: 2 - DNA, 2080 bp, Artificial
  Sequence of the glkA region amplified from the S. coelicolor M145 genome
aagatcgcggccggcgtggtcgacgaggaaggcaacatcctctcgacccacaaggtgccgacccccaccacacccgaggccatcgtggacgccatcgcctccgcggtcgagggggcgcgagtggggcacgagatcgtcgcagtgggcatcggcgccgccggatacgtcaaccgccagcgctccacggtctacttcgcgcccaacatcgactggcgccaggagccgctcaaggagaaggtcgaggcccgcgtcggcctcccggtcgtcgtggagaacgacgccaacgccgcggcctggggcgagtacaagttcggcggcggcaagggccaccgcaacgtcatctgcatcaccctcggcaccggcctcggcggcggcatcatcatcggcaacaagctgcgccgcggccacttcggcgtggccgcggagttcggccacatccgcatggtgccggacggcctgctgtgcggctgcggctcgcagggctgctgggagcagtacgcgtcggggcgggcgctggtgaggtacgccaagcagcgcgccaacgccacccccgagcgcgccgaggtgctgctcgccctcggcgacggcacccccgacggcatcgagggcaagcacatctcggtcgccgcccgccagggctgcccggtcgccgtcgactcctaccgggagctggcccgctgggccggggccggtctcgccgacctggcctcgctcttcgacccgtccgccttcatcgtcggcggcggcctctcggacgagggcgacctggtcctcgacccgatccgcaagtcctacaagcgctggctggtcggcggcaactggcgcccggtcgccgacgtgatcgccgcccaactcggcaacaaggccggcctggtcggcgccgccgacctggcgagagaacccgacccgatcatgtgacgatcacttcccgactccgggagctgcgctcacccgaggggcgcggggaactgcgcgaccagccacgacgaaccggcagtcgcccaggaaccccgcgccccgacccatgaggcgcctgatcatgcgttcccgtaaattgacggcatggcaacatccctcccggcgctccccaactcccgcacggaacccgacggttccgcggtcatccgcgtcctgagctacaacatccgctcactgcgcgacgacaccgacgccctcgcccgcgtcatcaaggcctgcgcgcccgacctggtcctcctccaggaggccccccgcttcttccgctggcgcaagaagatcacccggctcgccgccgccggcgatctggtcctgctctccggcggcggaaccgccgccggccccgcgctgctctgctccctgcgggccaccgtggagcgcaccgaggacgtgctgctcccgctcacccccggccggcaccgccggggcatcgccgccgccgtggtccggatcggcggcgcccgcatcggcgtactcagctcccacctgtccctggacgcggacgagcgccacgaacaggccgggctgctcctcgaccacctggccgccctcggcgtgaagcacgccgtggcgggcggcgacctgaacgagcgccccggcggccgcaccttccggcggctcggcgaagggctgcgcgactgctggaccgccgcgccctggggcggcgagtacaccttccccgcgaccgccccggaccgccggatcgacgcggtcttcgtcaccgagggcatcgaggtgctgggctgcggagtgccgtcgggactggccggggtggccgaggacgacctgagggcggccacggaccaccttccggtcctgaccgccctccgcgtacccgcgaccgcctgacccggggtccggccggccacccggctacaccaccgcgccccgccccggatcgtcgtcgtcctcgtcgcccgtccgcatccgcacgaccagggtggcgaagccgccgaggaaaccgccgatgctcagggtcgtcagccaccaggtcatgtcccagccgagcaccaccgccagcaggagcagcaccgggccgccgagcacgcccagccaggcgaacttcgaggtgggatcggtctcgggcagcggcggcggctcgggc

SEQ ID NO: 3 - protein, 317 aa, Artificial
  Strain of Streptomyces lividans and Streptomyces coelicolor (GLK1) from which glucokinases were derived
MGLTIGVDIGGTKIAAGVVDEEGNILSTHKVPTPTTPEAIVDAIASAVEGARVGHEIVAVGIGAAGYVNRQRSTVYFAPNIDWRQEPLKEKVEARVGLPVVVENDANAAAWGEYKFGGGKGHRNVICITLGTGLGGGIIIGNKLRRGHFGVAAEFGHIRMVPDGLLCGCGSQGCWEQYASGRALVRYAKQRANATPERAEVLLALGDGTPDGIEGKHISVAARQGCPVAVDSYRELARWAGAGLADLASLFDPSAFIVGGGLSDEGDLVLDPIRKSYKRWLVGGNWRPVADVIAAQLGNKAGLVGAADLAREPDPIM

SEQ ID NO: 4 - protein, 350 aa, Artificial
  Strain of Streptomyces coelicolor (GLK2) from which glucokinases were derived
APRVPTVGIDIGGTKVMAGVVDADGNILEKLRTETPDKSKSPKVVEDTIVELVLDLSDRHDVHAVGIGAAGWVDADRNRVLFAPHLSWRNEPLRDRIAGRLAVPVLVDNDANTAAWAEWRFGAGRGEDHLVMITLGTGIGGAILEDGQVKRGKYGVAGEFGHMQVVPGGHRCPCGNRGCWEQYSSGNALVREARELAAADSPVAYGIIEHVKGSIGDITGPMITELAREGDAMCVELLQDIGQWLGVGIANLAAALDPSCFVIGGGVSAADDLLIGPARDAFKRQLTGRGYRPEARIVRAQLGPEAGMVGAADLSRLVARRFRRAKRRRVERYERYERYAEARRESRESL

SEQ ID NO: 5 - protein, 319 aa, Artificial
  Strain of Bacillus subtilis from which glucokinases were derived
MDEIWFAGIDLGGTTIKLAFINQYGEIQHKWEVPTDKTGDTITVTIAKTIDSKLDEQKPKHIIKYIGMGAPGPVDMAAGVVYETVNLGWKNYALKNHLETETGIPAVIENDANIAALGEMKGAGDGAKDVILVTLGTGVGGGIIANGEILHGINGAGGEIGHICSIPEGGAPCNCGKTGCIETIASATGIVRIAKGKIANAKKTTRLKATEQLSARDVFEAAGENDEIALEVVDYVAKHLGLVLGNLASSLNPSKIVLGGGVSRAGELLRSKVEKTFRKCAFPRAAQAADISIAALGNDAGVIGGAWIAKNEWLKHQNC

SEQ ID NO: 6 - protein, 328 aa, Artificial
  Strain of Staphilococcus xylosus from which glucokinases were derived
MNKIILAADIGGTTCKLGIFDENLNRLSKWSIDTDTSDTTGYLLLKNIYDSFIQHIDKSDNTFSDVLGIGIGVPGPVNFETGEVNGAVNLYWKGTVNVRDIFKQFVDCPVYVDNDANVAALGEKHKGAGNGADDVVAITLGTGLGGGIISNGEIVHGHNGSGAEIGHFRVDHDQRFKCNCGKSGCIETVASATGVVNLVNFYYPKLTFKSSILQLIKDNKVSAKAVFDAAKAGDQFCIFITERVANYIAYLCSIVSVTSNPKYIVLGGGMSTAGLILIENIKTEYHNLTFTPAQQNTEIVQAQLGNDAGITGAAGLIYTYIIEKDGVK

SEQ ID NO: 7 - protein, 317 aa, Artificial
  Strain of Thermatoga maritima from which glucokinases were derived
MPKLKLIGVDLGGTTFSVGLVSEDGKILKKVTRDTLVENGKEDVIRRIAETILEVSDGEEAPYVGIGSPGSIDRENGIVRFSPNFPDWHNVPLTDELAKRTGKKVFLENDANAFVLGEKWFGAGRGHDHIVALTLGTGIGGGVVTHGYLLTGRDGIGAELGHVVVEPNGPMCNCGTRGCLEAVASATAIRRFLREGYKKYHSSLVYKLAGSPEKADAKHLFDAARQGDRFALMIRDRVVDALARAVAGYIHIFNPEIVIIGGGISRAGEILFGPLREKVVDYIMPSFVGTYEVVASPLVEDAGILGAASIIKERIGG

SEQ ID NO: 8 - protein, 301 aa, Artificial
  Strain of Synechocystius species from which glucokinases were derived
SIHREVIGIDIGGSSIKFGRFLSNGDCTESLVLPTPQPALPKAVYAQLAHGIDLLKNSACVAIGVGMPGPADAAGRVAQLAINLPQWHDIPLADWLEDHGQLPTVLENDANCAGLGEAWLGAGRDYRNFILLTLGTGVGGAVFLNGKLFTGPQGAGGELGLISIETEGYPCNSGNRGSLEQHASAQTLRREMNLTGLELAQLAAKGDAEAIAYWEKFGQRLGMGIANLVYVLTPEAVIIGGGLSASSSYFFPSMASEIEERVLLPSRRGLKILVAQLGNRAGMIGAARLAWQKLVYRTDGP

SEQ ID NO: 9 - protein, 323 aa, Artificial
  Strain of Deinococcus radians from which glucokinases were derived
AERAYLAALDVRTEGVAVLVSDLLGRVLAEASVPIDDHSGTGPAVEQAVSLVERAVKEAGADRLHTVGIGAPGLIDPAGGELRDSTGLPEWHRRLMAALQEKFPEARVGVENETNLAALAEQRDGAARDRDTFVLLWLGLGIGAAVVLDGTLRRGVSGGAGEIGFMPVPGTAGLPSATDCDGGFHSVAGAAAVTALAAEHGVTARPAGPEPHAAALVREAVRRSASGSDPAAERFLDALADRVVLGAAAVVSVLDPGCLVLAGEIGRAGADALADRVQHRLTRMSPLATEVRPSTLGGGAVLRGALLTARDRAQDDLFAPPER

SEQ ID NO: 10 - protein, 754 aa, Artificial
  ETR1 homologue derived from Lycopersicon esculentem
MGSLLRMNRLLSSIVESCNCIIDPQLPADDLLMKYQYISDFFIALAYFSIPVELIYFVKKSAVFPYRWVLVQFGAFIVLCGATHLINLWTFNMHTRNVAIVMTTPKALTALVSCITALMLVHIIPDLLSVKTRELFLKKKAAQLDREMGIIRTQEETGRHVRMLTHEIRSTLDRHTILKTTLVELGRTLALEECALWMPTRTGLELQLSYTLRHQNPVGLTVPIQLPVINQVFGTNHVVKISPNSPVARLRPAGKYMPGEVVAVRVPLLHLSNFQINDWPELSTKRYALMVLMLPSDSARQWHVHELELVEVVADQVAVALSHAAILEESMRARDLLMEQNVALDLARREAEMAVRARNDFLAVMNHEMRTPMHAIIALSSLLQETDLTPEQRLMVETILKSSNLLATLINDVLDLSRLEDGSLQLDIGTFNLHALFREVHSLIKPIASVKKLFVTLSLSSDLPEYVIGDEKRLMQILLNVVGNAVKFSKEGNVSISAFVAKSDSLRDPRAPEFFAVPSENHFYLRVQIKDTGIGITPQDIPNLFSKFTQSQALATTNSGGTGLGLAICKRFVNLMEGHIWIESEGLGKGSTAIFIIKLGIPGRSNEPKLPFMPRLPANHMQMTFQGLKVLIMDDNGFSRMVTKGLLVHLGCDVTTVSSRDECLRVVTHEHKVVIMDVSMQGIDCYEVAVVIHERFGKRHGRPLIVALTGNTDRVTKENCMRVGMDGVILKPVSVYKMRSVLSELLEHGVVLES

SEQ ID NO: 11 - protein, 738 aa, Artificial
  ETR1 homologue derived from Nicotiana tabacum
MDCNCFDPQWPADELLMKYQYISDFFIAVAYFSIPIELVYFVQKSAVFPYRWVLVQFGAFIVLCGATHLINLWTSTAHTRTLAIVMTTAKVLTAVVSCATALMLVHIIPDLLSVKTRELFLKNKAAELDREMGLIRTQEETGRYVRMLTHEIRSTLDRHTILKTTLVELGRTLALEECALWMPTPPGLELQLSYTLRHQNPIGFTVPIQLPVINQVFGTNRAVKISPNSPVARLRPAGKYMPGEVVAVRVPLLHLSNFQINDWPELSTKRYALMVLMLPSGSARQWHVHELELVEVVADQVAVALSHAAILEESMRARDLLMEQNVALDLARREAEMAVRARNDFLAVMNHEMRTPMHAIIALSSLLQETELTPEQRLMVETILKSSNLLATLINDVLDLSRLEDGSLQLDVGTFNLHVLFRKVLNLIKPIASVKNCLSRLTCLQICPEFAIGDEKRLMQILLNVVGNAVKFSKEGSVSISAVAAKSESLSDPRAPEFFPVQSENHFYLRVQVKDTGSGINPQDIPKLFCKFAQNQALATKSSGGTGLGLAISKRFVNLMEGHIWIESEGLGKGSTAIFIVKLGIPGRSNEPKLPFMPRLPANHMQMTFQGLKVLIMDDNGFSRMVTKGLLVHLGCDVTTVSSGDECLRVLTQEHKVVFMDVSIPGIDCYEVAVQIHEKFGKHHNRPLIVALTGNTDRVTKENCMRVGMDGVILKPVSVDKMRSVLSELLEHGVILQS

SEQ ID NO: 12 - protein, 740 aa, Artificial
  ETR1 homologue derived from Cucumis melo
MENCYCIEPQWPADELLMKYQYISDFFIALAYFSIPLELIYFVKKSAVFPYRWVLVQFGAFIVLCGATHLINLWTFTMHSRTVAVVMTTAKVLTAVVSCATALMLVHIIPDLLSVKTRELFLKNKAAELDREMGLIRTQEETGRHVRMLTHEIRSTLDRHTILKTTLVELGRTLALEECALWMPTRTGLELQLSYTLHQQNPVGYTVPINLPVISQVFSSNRALKISPNSPVASLRPRAGRYVAGEVVAVRVPLLHLSNFQINDWPELSTKRYALMVLMLPSDSARQWRVHELELVEVVADQVAVALSHAAILEESMRARDLLMEQNVALDLARREAETAIRARNDFLAVMNHEMRTPMHAIIALSSLLQETELTPEQRLMVETILKSSNLLATLINDVLDLSRLEDGSLQLDIGTFNLHAVFKEVLNLIKPVTLVKKLSLTLHLGPDLPVFAVGDEKRLMQAILNVVGNAVKFSKEGSISISAIVAKSETFREIRVPDFHPVPSDSHFYLRVQVKDTGSGISPQDIPKLFTKFAQTTVGPRNSGGSGLGLAICKRFVNLMEGHIWLESEGLGKGCTATFIVKLGIADQSNESKLPYTSKIHENSIHTSFPGLKVLVMDDNGVSRSVTKGLLVHLGCEVTTAGSIEEFLRVVSQEHKVVFMDICTPGVDGYELAIRIREKFAKCHERPFMVVLTGNSDKVTKESCLRAGMDGLILKPVSIDKMRSVLSELIERRVLFETS

SEQ ID NO: 13 - protein, 738 aa, Artificial
  ETR1 homologue derived from Arabidopsis thaliana
MEVCNCIEPQWPADELLMKYQYISDFFIAIAYFSIPLELIYFVKKSAVFPYRWVLVQFGAFIVLCGATHLINLWTFTTHSRTVALVMTTAKVLTAVVSCATALMLVHIIPDLLSVKTRELFLKNKAAELDREMGLIRTQEETGRHVRMLTHEIRSTLDRHTILKTTLVELGRTLALEECALWMPTRTGLELQLSYTLRHQHPVEYTVPIQLPVINQVFGTSRAVKISPNSPVARLRPVSGKYMLGEVVAVRVPLLHLSNFQINDWPELSTKRYALMVLMLPSDSARQWHVHELELVEVVADQVAVALSHAAILEESMRARDLLMEQNVALDLARREAETAIRARNDFLAVMNHEMRTPMHAIIALSSLLQETELTPEQRLMVETILKSSNLLATLMNDVLDLSRLEDGSLQLELGTFNLHTLFREVLNLIKPIAVVKKLPITLNLAPDLPEFVVGDEKRLMQIILNIVGNAVKFSKQGSISVTALVTKSDTRAADFFVVPTGSHFYLRVKVKDSGAGINPQDIPKIFTKFAQTQSLATRSSGGSGLGLAISKRFVNLMEGNIWIESDGLGKGCTAIFDVKLGISERSNESKQSGIPKVPAIPRHSNFTGLKVLVMDENGVSRMVTKGLLVHLGCEVTTVSSNEECLRVVSHEHKVVFMDVCMPGVENYQIALRIHEKFTKQRHQRPLLVALSGNTDKSTKEKCMSFGLDGVLLKPVSLDNIRDVLSDLLEPRVLYEGM